(19) United States
(12) Patent Application Publication
(10) Pub. No.: US 2025/0213328 A1
(43) Pub. Date: Jul. 3, 2025

(54) SYSTEMS AND METHODS FOR REMOTE PROGRESS SCANNING

(71) Applicant: ALIGN TECHNOLOGY, INC., San Jose, CA (US)

(72) Inventor: Ofer SAPHIER, Rehovot (IL)

(73) Assignee: ALIGN TECHNOLOGY, INC., San Jose, CA (US)

(21) Appl. No.: 19/005,731

(22) Filed: Dec. 30, 2024

Related U.S. Application Data
(60) Provisional application No. 63/617,193, filed on Jan. 3, 2024.

(51) Int. Cl. A61C 7/00; A61B 1/00; A61B 1/24; A61C 9/00; A61C 13/34; G06T 7/73; G06T 17/00; G06T 19/20

(52) U.S. Cl. CPC A61C 7/002; A61B 1/00172; A61B 1/24; A61C 9/0053; A61C 13/34; G06T 7/73; G06T 17/00; G06T 19/20; G06T 2210/41; G06T 2210/56

(57) ABSTRACT

A system for scanning a dentition may include an intraoral scanner, a processor, and memory storing instructions that when executed by the one or more processors may cause the system to receive point cloud data of a patient's detention, and generate a plurality of possible models of the patient's dentition based on the received point cloud data by manipulating an aspect of the point cloud data multiple times, for each manipulation of the point cloud data, generating a possible model of the patient's dentition to generate the plurality of possible models of the patient's dentition and determine a position variance between positions of respective dental features of the patient's detention between each of the possible models of the of the patient's dentition. The system may also output a depiction of the scanned location on the patient's detention based the measured range of possible models.

### BACKGROUND

This application claims the benefit under 35 U.S.C. § 119(e) of U.S. Provisional Patent Application No. 63/617,193, filed Jan. 3, 2024, and titled “SYSTEMS AND METHODS FOR REMOTE PROGRESS SCANNING,” which is hereby incorporated, in its entirety, by this reference.

The landscape of medical practice is evolving, with a shift towards telemedicine—the practice of remotely diagnosing and treating patients. This approach offers healthcare providers the ability to assess a patient's needs and, in many cases, provide treatment recommendations without the logistical complexities and associated risks that often come with in-person healthcare interactions. However, while telemedicine has made significant strides in various medical specialties, the realm of dental care still lags behind in several key aspects.

In the world of dental care, there is a persistent reliance on in-person consultations and traditional diagnostic methods. This requirement for physical presence can encompass a wide range of dental needs, including initial assessments, the procurement of accurate diagnoses for a variety of oral conditions, the development and communication of comprehensive treatment plans, and the follow-up monitoring of a patient's progress throughout their treatment. Such reliance on in-person consultations, while effective in many scenarios, presents a multitude of challenges in world.

The geographical inaccessibility of dental care can be a significant impediment for those living in remote or underserved areas. These individuals may incur substantial time and expense to receive routine care. The inconvenience and financial burden can deter people from seeking dental treatment.

Tele-dentistry is emerging as a promising solution, aiming to bridge the gap between traditional dental practices and modern, tech-driven healthcare. By harnessing the power of telecommunications and digital technology, tele-dentistry offers the potential to provide comprehensive dental services remotely, ensuring that patients can receive timely and effective care, regardless of their location or circumstances.

While telemedicine is transforming the broader healthcare landscape, there remains substantial room for improvement in the field of dental care. The limitations of in-person dental consultations become particularly evident during crises, periods of physical inaccessibility, and for individuals facing mobility challenges. The ongoing development and adoption of tele-dentistry hold the promise of making dental care more accessible, efficient, and patient-centered, ultimately contributing to better oral health outcomes for all.

Existing dental progress tracking is less than ideal in a number of ways. For example, they rely on the use of expensive dental office 3D scanners or less than ideal cell phone or consumer cameras. While dental office 3D scanners can provide detailed and accurate representations of a patient's dental structure, their high price tags, large size, and the specialized expertise needed to operate make them unsuitable for remote and patient use.

Reliance on consumer-grade technology, such as cell phone cameras, or even less advanced consumer cameras are also less than ideal. These devices, while ubiquitous, are not optimized for capturing precise dental images. They may lack the necessary resolution, lighting control, and image stability required for accurate dental assessments. As a result, the data they produce may be suboptimal, leading to inaccuracies in tracking a patient's progress throughout their treatment.

### SUMMARY

As will be described in greater detail below, the present disclosure describes various systems and methods for remote progress tracking and imaging to determine the position of a patient's teeth. The disposure herein provides improvements to the computing systems and methods for generating accurate dental models, treatment planning, and dental treatment.

### INCORPORATION BY REFERENCE

All patents, applications, and publications referred to and identified herein are hereby incorporated by reference in their entirety, and shall be considered fully incorporated by reference even though referred to elsewhere in the application.

### BRIEF DESCRIPTION OF THE DRAWINGS

A better understanding of the features, advantages and principles of the present disclosure will be obtained by reference to the following detailed description that sets forth illustrative embodiments, and the accompanying drawings of which:

FIG. 1 shows a method for dental treatment using remote progress scanning of a patient's teeth, in accordance with some embodiments.

FIGS. 2A and 2B show models of a patient's teeth, in accordance with some embodiments.

FIG. 3 shows method of remote progress scanning of a patient's teeth, in accordance with some embodiments.

FIG. 4 shows a display of a dentition and scanner, in accordance with some embodiments.

FIG. 5 shows a display of feedback on a dentition, in accordance with some embodiments.

FIG. 6 shows a method for dental treatment using remote progress scanning of a patient's teeth, in accordance with some embodiments.

FIG. 7 shows a system scanning a dentition using an at-home intraoral scanner, in accordance with some embodiments.

FIG. 8 illustrates an exemplary tooth repositioning appliance or aligner that can be worn by a patient in order to achieve an incremental repositioning of individual teeth in the jaw, in accordance with some embodiments.

FIG. 9 illustrates a tooth repositioning system, in accordance with some embodiments.

FIG. 10 shows a method of orthodontic treatment using a plurality of appliances, in accordance with embodiments.

FIG. 11 shows a method for digitally planning an orthodontic treatment, in accordance with embodiments.

FIG. 12 shows a simplified block diagram of a data processing system, in accordance with embodiments.

FIG. 13 shows a block diagram of an example computing system capable of implementing one or more embodiments described and/or illustrated herein, in accordance with some embodiments.

FIG. 14 shows a block diagram of an example computing network capable of implementing one or more of the embodiments described and/or illustrated herein, in accordance with some embodiments.

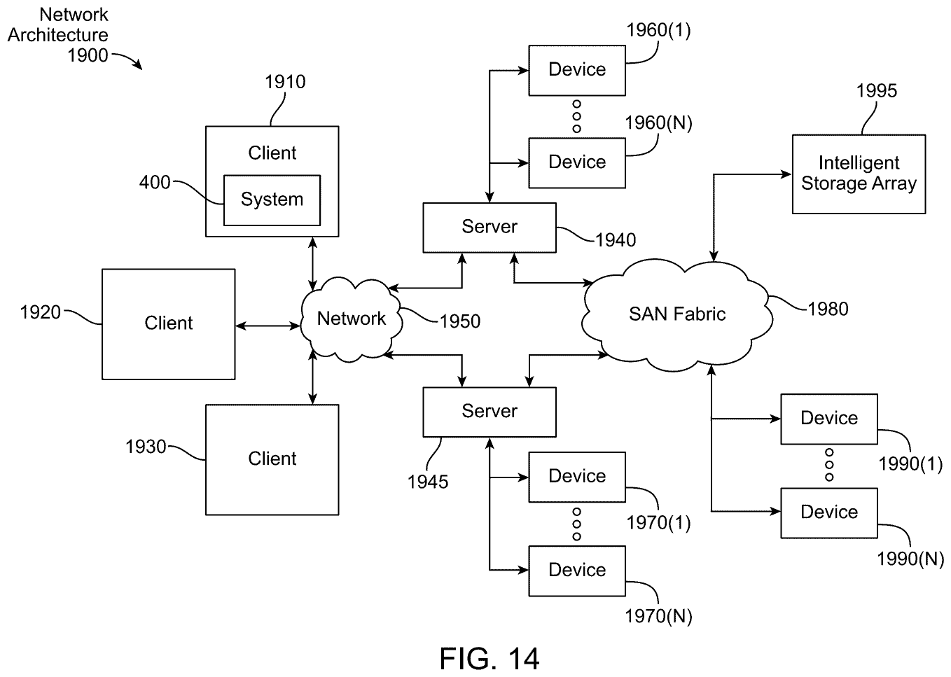

### DETAILED DESCRIPTION

The following detailed description provides a better understanding of the features and advantages of the inventions described in the present disclosure in accordance with the embodiments disclosed herein. Although the detailed description includes many specific embodiments, these are provided by way of example only and should not be construed as limiting the scope of the inventions disclosed herein.

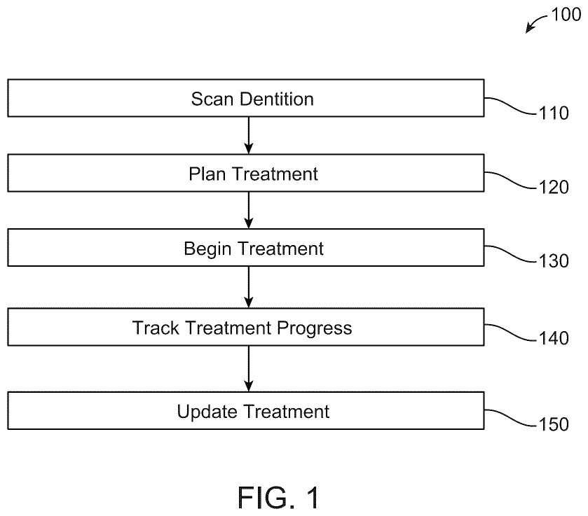

FIG. 1 shows a method 100 for dental treatment using remote progress scanning of a patient's teeth. The method may include scanning a dentition of a patient at block 110, generating a treatment plan at block 120, beginning treatment at block 130, tracking treatment progress at block 140, and updating the treatment plan at block 150.

At block 110, in some embodiments, the patient's dentition is scanned with the three-dimensional intraoral scanner in order to generate a three-dimensional model of the patient's dentition. The three-dimensional model of the patient's dentition may be segmented to include individually segmented teeth representing each of the patient's teeth of their upper and lower arches.

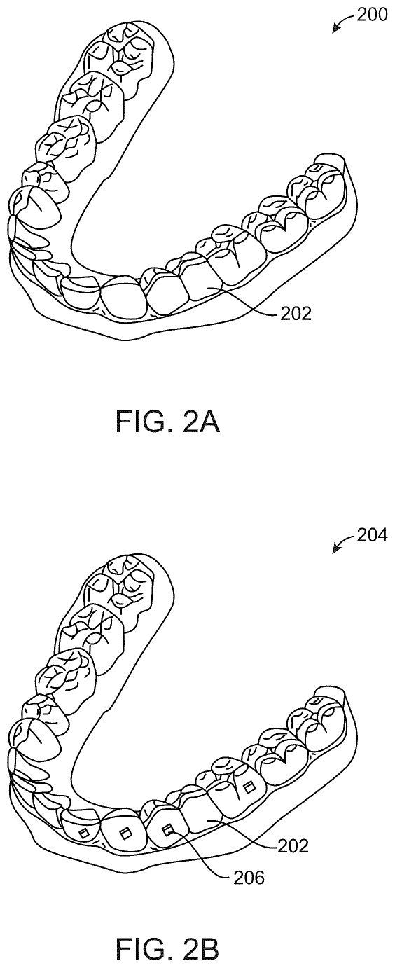

For example, FIG. 2A depicts a three-dimensional model 200 of an arch of a patient's dentition. The three-dimensional model may include the shape, position, and orientation of the patient's teeth 202, and other dental and oral tissues within the oral cavity. For example the three-dimensional model may include the patient's gingiva, also referred to as gums, tongue, lips, and other anatomy. The initial scan of the patient's teeth 202 includes the position and orientation of the patient's teeth at the time of the scan.

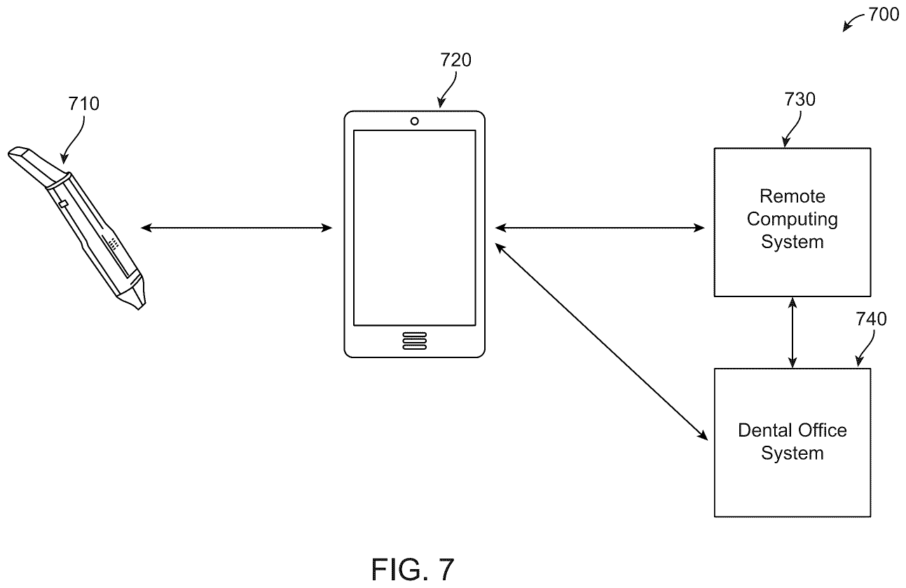

The initial scan may be performed by a dental professional, such as at a dental office, using a high resolution intraoral scanner as part of a dental office system 740, see FIG. 7.

At block 120 a treatment plan may be generated. An assessment may be made as to desired final positions of the patient's teeth based on the initial position of the patient's teeth obtained from the intraoral scan. A series of intermediate tooth positions of the patient's teeth may be generated to incrementally move the teeth through a series of stages from the initial positions towards the final positions.

FIG. 2B depicts a three-dimensional model 204 of an arch of the patient's dentition generated as part of the treatment planning process. The three-dimensional model may include the shape, position, and orientation of the patient's teeth 202 during a stage of treatment, and other dental and oral tissues within the oral cavity. In some embodiments individual attachments or other anchoring elements 206 may be used on teeth 202 with corresponding receptacles or apertures in the appliance so that the appliance can apply a selected force on the tooth. In some embodiments, a reference model as discussed herein may include the position and orientation of the patient's teeth and one or more attachments located thereon.

In some embodiment, the 3D model of the patient's dentition may be sent to a remote computer system 730, see FIG. 7. The remote computing system may generate the treatment plan and carry out the process of block 120.

At block 130 treatment may begin. Dental appliances may be fabricated based on the intermediate positions of the patient's teeth in order to move the teeth from the initial positions towards the final positions. A patient then wears each of the dental appliance for a period of time, for example 10 days to two weeks, during which time the dental appliance applies forces to the patient's teeth to move the teeth from a first position at the beginning of the treatment stage towards a second position at the end of the treatment stage. Each appliance is worn in succession in order to move the patient's teeth.

However, treatment may not progress as expected. Sometimes a patient's compliance may not be as expected. A patient may not wear the dental appliances throughout the day, for example they may take them off before eating and forget to put them on after completing their meal. Such lack of compliance may lead to teeth positions lagging their expected positions. Sometimes teeth provide more or less resistance to movement than expected. This may result in teeth moving slower or faster than expected. Differences in compliance and tooth resistance may result in a treatment going off-track, such that the actual position of the patient's tooth during a given stage of treatment may deviate from the expected position to a degree that an appliance may not fit the patient's teeth or may otherwise not provide the desired movement for stage.

Doctors may monitor the progress of the patient's treatment in order to anticipate or determined that a patient's treatment has gone off-track or may be progressing towards off-track so that they can provide intervention in order to bring the treatment back on track or to generate a new treatment plan to treat the patient's teeth.

At some point during treatment the doctor may decide to assess a patient's progress, for example at each stage of the patient's treatment their doctor may request the patient to take one or more photos of the patient's teeth, guided by the artificial intelligence systems and methods discussed herein.

At block 140 progress of the treatment and in particular, progress tracking, may include comparing the actual movement and position of a patient's teeth during orthodontic treatment as compared to the expected movement and position of the patient's teeth during orthodontic treatment. The comparison may include determining the positional deviation or error between the actual position of the patient's teeth and the expected position of the patient's teeth based on a three-dimensional model of the patient's teeth for the particular stage of treatment.

The progress scanning may occur at a remote location such as a location remote from a dental office and may be performed using a lower quality scanner 710 of FIG. 7, as compared to the scanner used to generate the initial treatment plan and 3D model. The lower quality scanner which may be referenced as a home intraoral scanner, may have a lower resolution and lower accuracy than the scanner used to generate the initial treatment planning a 3D model, which may be referred to as the reference scan or reference model. For example, the lower resolution may include using a lower density of data points when generating scan, such as between 0.5 and 4 megapixels, and preferably between 0.25 and 2 megapixels, for cameras and between six and 15 lines for structured light system. In some embodiments, the structured light system may capture less than 150 depth points within the field of view, preferably between 50 and 100 depth points within the field of view. As another example, the lower accuracy of the scanner may include using the scanner having a lower accuracy in the determination of the depth location of a data point.

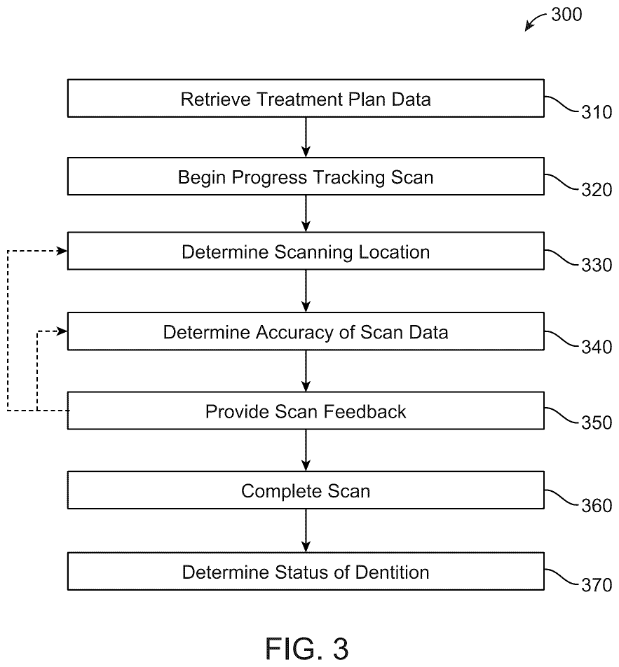

When using a lower accuracy 3D scanner the data generated by the home intraoral scanner may be analyzed to detect when enough data from the home intraoral 3D scanner has been generated in order to determine the position of the particular tooth. The various processes and systems for performing the progress tracking scan are shown and described herein with reference to FIGS. 3-6.

At block 150 the treatment plan may be updated based on the position and orientation of the patient's teeth in the progress in progress scan. For example, a doctor may remotely assess the patient's treatment progress and in the rare cases when the patient's progress becomes so off-track as to warrant a revised treatment plan, the progress scan and the reference scan may be used to prescribe a secondary order for an off-track patient using only their primary treatment plan data, including the reference scan, without rescanning the patient in the dental office. For example, a new treatment planning process may begin based on the updated position of the patient's teeth from the progress scan in order to generate a revised treatment plan to move the patient's teeth from the new current position to the desired final position. In some embodiments, the revised treatment plan may move the teeth to a different, new desired final position.

In some embodiments, patient's tooth movements are in the correct direction but lagging behind in position, the updated treatment may include instructions for the patient to continue wearing an aligner or aligners for a particular stage of treatment for an additional time period. In some embodiments, for example wherein the patient's tooth movements are in the correct direction but progressing faster in their position, the updated treatment may include instructions for the patient to proceed to the next aligner or aligners in a next stage of treatment sooner than the time period specified in the original treatment plan.

In some embodiments, such as wherein the tooth positions are unexpected such as significantly lagging in movement or ahead of movement, or have deviated from the expected path, the systems and methods herein may provide for and may notify the doctor of the situation and recommend a in person evaluation of the patient's dentition.

In some embodiments, such as for controlled and understood deviations from the plan, an updated treatment plan may be generated to create treatment stages and corresponding aligners that move the patient's teeth back to their planned trajectory and movement path such as to a position and orientation in the original treatment plan. From there the original treatment plan and corresponding aligners may be used to treat the patient's dentition.

In some embodiments, a patient may be provided with a set of aligners for many stages of treatment, for example, three or more stages of treatment. The treatment tracking process of block 140 may occur at one of the stages of treatment, such as at the end of stage three, after wearing three aligners. If the treatment tracking shows that the teeth are off track, at block 150, the treatment may be updates with additional stages of treatment to move the teeth from their current position to the expected position for the next stage of the original treatment stages. This allows the patient to resume using the originally provided aligners after wearing the additional aligners for the additional stages to move the teeth from their current position to the expected position for the original treatment stages.

In some embodiments, a patient may be provided with a first set of aligners for many stages of treatment, for example, three or more stages of treatment. After wearing the first set of aligners, or during the final stage of the first set of aligners, the patient's progress may be tracked and the treatment plan may be updated using the current position of the patient's teeth as an initial position and a revised treatment plan may be generated to move the patient's teeth to the final position from the current position. A second set of aligners may be provided to the patient and the process may be repeated, wherein after wearing the second set of aligners, or during the final stage of the second set of aligners, the patient's progress may be tracked and the treatment plan may be updated using the current position of the patient's teeth as an initial position and a revised treatment plan may be generated to move the patient's teeth to the final position from the current position. A third set of aligners may be provided to the patient. This process may repeat until the patient's teeth are in their desired positions. This process reduces wasted aligner fabrication time and materials.

FIG. 3 shows a method 300 of remote progress scanning of a patient's teeth. The method may include retrieving a treatment plan at block 310, beginning a progress tracking scan at block 320, determining a location of the scan at block 330, determining an accuracy of the scan data at block 340, providing feedback at block 350, completing the scan at block 360, and determining the status of the detention at block 370.

At block 310 the patient's treatment plan may be retrieved. In some embodiments retrieving the treatment plan may include retrieving a three-dimensional model of the patient's dentition for a particular stage of treatment. If the treatment plan included attachments to aid in moving the patient's teeth or for other reasons, then the retrieved three-dimensional model of the patient's dentition for the particular stage of treatment in the treatment plan may include the three-dimensional models of the attachments.

In some embodiments, the treatment plan may be retrieved and provided to a patient's device 720 such as a patient's smart phone. For example, when processing of a progress scan takes place on the patient's device, the treatment plan and or associated three-dimensional model for a stage of the patient's treatment may be received on the patient's device from a remote computing system, such as remote computing system 730, which may be a system for generating and/or storing treatment plan information.

In some embodiments, processing of the progress scan as discussed herein may be performed remotely such as at a remote computer or server, such as remote computing system 730, which may include a plurality of remote and interconnected systems. In such embodiments the treatment plan may be retrieved by the remote computer or server from the data store on the remote computer or server or on another remote computer or server. In some embodiments, the 3D model of the patient's dentition for a particular stage of treatment may be retrieved onto both the remote computer or server and/or the patient's device.

In some embodiments, retrieving the treatment plan data may include retrieving attachments on a patient's dentition according to a stage of treatment and placing those attachments onto a generic model of an upper and/or lower arch of the generic dentition model.

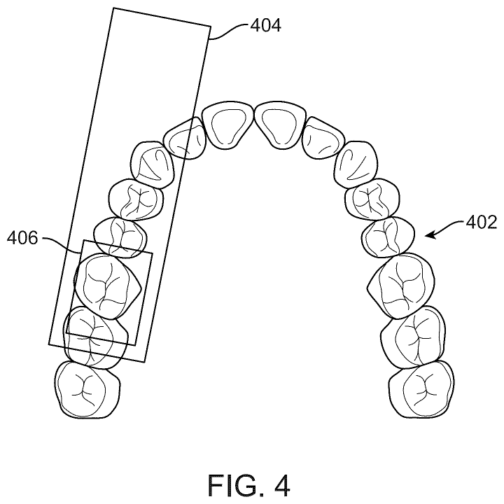

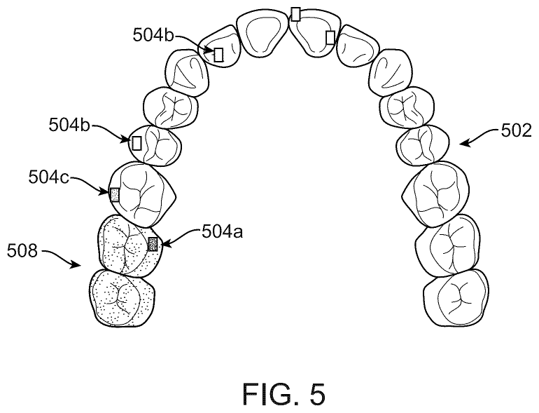

FIG. 4 depicts a model 402 of the patient's dentition, which may be a three-dimensional model for a stage of treatment or a generic dentition model without attachments. FIG. 5 depicts a model 502 of the patient's dentition, which may be a three-dimensional model for a stage of treatment or a generic dentition model with attachments.

At block 320, a progress tracking scanning process may begin. The progress tracking scanning process may be performed using a three-dimensional intraoral scanner 710 with a limited field of view. The field of view of the three-dimensional intraoral scanner may be one tooth, two teeth, three teeth, or four teeth. Preferably the field of view of the three-dimensional intraoral scanner during the scanning process is between two and four teeth, such as between 15 mm and 35 mm wide to capture a 15 mm to 35 mm length of the arch, in order to capture the relative positions of at least two adjacent teeth, but less than five adjacent teeth. The 3D scanner may generate images using smooth illumination or structured light illumination or both in order to generate a plurality of images from which three-dimensional point clouds of the surface of the patient's dentition and oral cavity are generated.

In some embodiments, such as for structured light scanning, the three-dimensional point clouds generated from each frame of data may be insufficient or sparse such that they may not be readily combined or stitch together in order to generate a full three-dimensional model of the patient's dentition. Instead, as discussed herein multiple point cloud data from the at home 3D intraoral scanner may be compared to the reference 3D model in order to determine the relative positions of the patient's teeth. The sufficiency of the data for use in comparing to the reference 3D model may be determined locally such as on the patient's smart phone 720, or other device, or the data may be sent to a remote computing system 730, which may be a 3D processing system for processing the 3D data.

In some embodiments, a first processor located on the scanning device. The first processor may receive image data from, for example, an image sensor on the scanning device. The image data may include image attributes and locations, such as coordinates associated with the image attributes. Image attributes may include information, such as depth data or information from which depth data may be derived, such as intensity, phase or phase offset, etc. The first processors may provide the image data to a second system, such as the mobile device or remote system discussed herein for further processing, such as the generation of the point cloud data from the image data.

Feedback may be provided to the user during the remote progress tracking scanning process. For example, FIG. 4 depicts an image that may be displayed on a patient's device such as a mobile phone. The image includes a model of a dentition 402 which may be a model of the patient's dentition or a generic dentition model along with a position and/or orientation of an intraoral scanner 404 and its field of view 406. The feedback shown in FIG. 4 user during the scanning process. In order to provide the feedback shown in FIG. 4, the system may determine the position and/or orientation of the intraoral scanner 404 and the field of view 406. The feedback may be provided by the patient's device. In some embodiments, feedback data may be provided by the remote computing system to the patient's device. The patient's device may use the feedback data to generate feedback for the patient.

At block 330 the process may include determining the scanning location. The process of block 330 may occur on the patient's device 720 based on data received from the scanner 710 or the process of block 330 may occur on a remote computing device 730 based on data received from the scanner 710, such as via the patient's device 720. Determining the scanning location may include determining which tooth or teeth or other dental or oral structure is within the field of view of the intraoral scanner. In some embodiments, the position and/or field of view of the intraoral scanner provided in the feedback, such as shown in FIG. 4, may be determined based on the scanning location.

In some embodiments the scanning location may be determined by fitting the scan data to a reference three-dimensional model. The reference three-dimensional model may be the retrieved model from the treatment plan for the stage of treatment during which the scan is occurring. In some embodiments, the scanning location may be determined by reference to shape descriptors. For example shape descriptors of the may be compared to shape descriptors of the retrieved three-dimensional model. Based on this comparison the location of the scan data on the dentition of the patient may be determined.

In some embodiments the intraoral scanner may capture image data used to determine the location of the scanner relative to the patient's teeth. For example, an image of the patient's teeth may be input to train a deep neural network in order to determine the teeth within the image. For example, the deep neural network may have been trained on annotated or marked images of the patient's teeth. The images may have been marked to indicate the tooth or teeth within the image.

In some embodiments, anatomical features such as the tooth gumline and the boundary between two adjacent teeth may be used in order to determine the position of the intraoral scanner relative to the patient's dentition. For example, the anatomical features within the field of view of the intraoral scanner may be compared to the shape of the anatomical features of the patient's dentition in order to match the anatomical features between the two data sets and determine the position of the intraoral scanner.

In some embodiments, a data set of silhouettes of the patient's teeth based on the retrieved treatment plan data may be generated various angle viewpoints by projecting the 3D model onto a 2D image plane at each of the view points from which the silhouette is determined. The silhouettes of the patient's teeth may be extracted from the progress scanning data and compared to the data set of silhouettes generated based on the treatment plan data in order to determine where the intraoral scanner.

In an in office intraoral scanner, a 3D surface model of the patient's dentition is generated as the scanner scans the teeth. This process includes stitching height maps or point clouds of the surface of the patient's teeth generated by the 3D scanner to one another. The process described herein however may use the 3D scanner with insufficient's fidelity to stitched point clouds to one another or may generate such sparse three-dimensional data that they are not stitched to one another. However, because the reference scan data or model is available, the location on the dentition of the data from the progress tracking three-dimensional scan can be determined based on a comparison of the data to the reference model.

Once the location of the intraoral scanner and/or its field of view is determined, feedback such as shown in FIG. 4 may be provided to the user.

At block 340 the progress scanning process continues by gathering sets of point cloud data or other image data and determining the accuracy, such as the accuracy of the depth data within the point cloud or other image data. The accuracy of the point cloud or other image data gathered by the progress scan may be determined in many ways.

In some embodiments, a first processor located on the scanning device. The first processor may receive image data from, for example, an image sensor on the scanning device. The image data may include image attributes and locations, such as coordinates associated with the image attributes. Image attributes may include information, such as depth data or information from which depth data may be derived, such as intensity, phase or phase offset, etc. The first processors may provide the image data to a second system, such as the mobile device or remote system discussed herein for further processing, such as the generation of the point cloud data from the image data.

The process of block 340 may occur on the patient's device 720 based on data received from the scanner 710 or the process of block 340 may occur on a remote computing device 730 based on data received from the scanner 710, such as via the patient's device 720. The process may include measuring a range of possible solutions, which may be possible positions of the surface data and corresponding teeth represented in the point cloud data, based on the received point cloud data by manipulating an aspect of the point cloud data multiple times, for each manipulation of the point cloud data, calculating a possible model based on the altered point cloud data to generate a plurality of possible models, and computing a variance between each of the calculated possible models of the generated plurality of possible models.

In some embodiments such as for structured light data that generates a point cloud that is insufficient for accurately stitching between point cloud data sets, point cloud data may be gathered until the accuracy is estimated to be above a threshold for determining the relative position of two or more teeth within a point cloud data.

In some embodiments, a relative position between two known teeth of known shapes can be described with six degrees of freedom, three degrees of positional freedom and three degrees of rotational freedom, such as the difference between the position and orientation of one tooth with respect to the other tooth. Similarly, a relative position between three teeth of known shapes can be described with twelve degrees of freedom, three degrees of positional freedom and three degrees of rotational freedom for each of two teeth relative to that of a third or three degrees of positional freedom and three degrees of rotational freedom between one tooth an a second tooth and an additional three degrees of positional freedom and three degrees of rotational freedom between the second tooth and a third tooth.

The relative position of the teeth may be computed by aligning the point clouds generated by the progress scanning process to the segmented teeth surfaces and attachments in reference model. A range of possible alignments and corresponding tooth shapes may be measured. During the alignment process accuracy of the gathered data for use in determining the degrees of freedom of the relative tooth positions may be determined using an optimization algorithm such as the iterative closest point algorithm.

The Iterative Closest Point (ICP) algorithm may be used to align and register two sets of 3D or 2D points to find their optimal spatial transformation (translation and rotation) that minimizes the differences between corresponding points in the two sets, sometime referred to as stitch two sets of point clouds together.

A first part of the process may include starting with an initial estimate of the transformation between the two point sets, such as the progress scan set and the reference model. This initial estimate can be obtained through various means, such as a pre-alignment process, which may include manual alignment or rough estimation or randomly. Random pre-alignment may include starting at a few random initial transforms of the point cloud data and evaluating how close each of the transforms are to the reference model. If one of the transforms results in the point cloud being within a threshold of the reference model, that transform may be used as a starting point. If none are within a threshold or multiple are within a threshold, then the closest transform may be used. In some embodiments, a set of possible orientations and translations (transforms) may be used and the closest of the set selected as the starting point.

In some embodiments, white light color images form the 3D scanner may be used to identify the teeth within the field of view and an initial transform. These images can be compared to white light color images captured during the initial scan, such as the scan captured at block 110 in order to determine an initial starting point.

Next, for each point in the source point set of the progress scan data, the closest point in the target point set, such as the reference model, is found. This step establishes correspondences between points in the two sets. In some embodiments, the Euclidean distance may be used.

Next, weights may be assigned to the point correspondences based on their proximity. Points that are closer together may be given higher weights, while points that are farther apart have lower weights. This step may help in giving more importance to accurate correspondences. In some embodiments, each point pair or correspondence may be equally weighted. In some embodiments, weights may be assigned based on the compatibility of the surface normal at the point locations, such as by the dot product of the vectors representing the normal. In some embodiments, weights may be assigned based on expected scanner noise and uncertainty, for example, points at locations on a surface at different orientations with respect to the scanner may be given different weights, based on their orientation. Points at locations more perpendicular to the scanner may be given weights that are different than points at locations on a surface that are less perpendicular.

Using the correspondences, either weighted or unweighted, an estimate of a transformation (usually a combination of translation and rotation) that aligns the source cloud with the target cloud is generated The estimated transformation to the source cloud may then be updated, transforming it closer to the alignment of the target cloud.

The alignment estimate may be checked to determine whether the alignment estimate has converged to a position and orientation within a threshold of alignment. This may be based on a convergence criteria, such as the change in transformation parameters falling below a certain threshold between iterations.

If the alignment threshold criteria are not met, then the process repeats. In some embodiments, the point cloud data is iteratively aligned with the reference model until the threshold is met. If the threshold is not met such as after a set number of iterations, additional point cloud data sets may be generated from the at-home intraoral scan or as part of the progress scanning process. n some embodiments the additional set or sets of point cloud data may be gathered, the data may be aligned, and the alignment may be checked against the threshold.

Although the iterative closest point method is described herein other alignment methods may be used. The other alignment methods may include an optimization procedure that attempts to minimize a loss function such as the minimal some of distances between the point cloud data of the progress tracking scan and the reference data from the treatment plan. In some embodiments, such as those with a significant number of degrees of freedom, a nonlinear optimization may be used.

Each of the aligned or optimized point clouds may be manipulated to determine if the optimized point cloud is in a correct alignment with the reference model. For example, the point cloud data may be manipulated by adding noise, altering the optimization starting point, deleting points in the point cloud data, or other methods, as described herein. A variance between each of the calculated alignments, which may be a possible 3D model shape may be determined. If the variance is small, such as within a predefined accuracy, then that location of the dentition is considered to have been scanned to a sufficient accuracy.

One or more of many threshold criteria for determining whether the data is sufficiently accurate for determining the relative positions of two teeth may be used. For example, the optimization algorithm such as the iterative closest point algorithm may be run from multiple different starting points in the range of the solutions from each starting point may be determined. If for all or a sufficient number of starting positions, the solutions range is below a threshold of accuracy of the degrees of freedom then sufficiently accurate scan data has been captured. In some embodiments, the optimization may be run multiple times but noise may be added to the point cloud data. If each of the data sets with noise added converge to within a range of accuracy sufficient for determining the relative positions of two or more teeth then sufficiently accurate scan data has been captured.

In some embodiments, the optimization may be performed multiple times, but data points randomly removed from the point cloud data for each run of the optimization. If each run of the optimization is within a range or predetermined threshold of accuracy then the desired accuracy has been achieved. In some embodiments, a combination of the optimizations described herein may be performed and, if they all converge to within a threshold of accuracy sufficient for determining the relative position of two or more teeth, such as a location and rotation about the 6 degrees of freedom, then a sufficient number of the data sets have been captured.

In some embodiments, after optimizing the degrees of freedom for multiple teeth within the point cloud, the process may include aligning the point cloud to the reference model or the reference model to the point cloud on a tooth by tooth basis. For example, teeth may not be in the same relative positions in the point cloud data as in the reference model, such as when a patient's treatment is off track. After an initial alignment between the point cloud and the reference model, individual tooth transformations maybe made. In some embodiments, a single tooth of the reference model may be aligned with the point cloud. In some embodiments, the point cloud data may be split or segmented, wherein each point in the point cloud is assigned to a particular tooth, such as based on tooth in the reference model they are closes to. Each tooth from the reference model may then be aligned with the point cloud. The alignment may include a transformation (translation and rotation, each in 3 three degrees of freedom) such that the points in the reference model most closely align with the points in the point cloud to generate an updated model of the patient's dentition.

In some embodiments, alternatively or in addition to the optimizations discussed above, a machine learning method may be used to determine whether the received scan data from the progress scanning processes sufficient to determine the relative positions of the patient's teeth. The machine learning model may be trained using input past successful and/or unsuccessful optimization data. Machine learning model may be trained using features or aspects of the successful and/or unsuccessful optimization data including the number of data points for each tooth for each frame of gathered point cloud data, an estimate of the noise within the three-dimensional data in the data points, the number of scans collected as part of the scanning process for a particular tooth or set of teeth, the surface region of the teeth within the scan, in the shape of the tooth or teeth being scanned. The neural network or other machine learning model may be trained to determine whether sufficiently accurate data has been gathered based on these features and aspects. The model may make the accuracy determination without reference to the reference model, but simply based on the features and aspects of the data collected. A higher threshold for accuracy determination using the machine learning or neural network models may be used as compared to the optimization models discussed herein.

In some embodiments, such as for high density or higher density structured light data such as structured light data with point clouds that are sufficiently dense to allow stitching between point cloud data, a larger point cloud or surface model may be stitched together between the scanned point clouds and the methods described above may be applied to the stitched point clouds. However, even the stitched point clouds discussed herein may be of a lower accuracy and density and the point clouds used to build the reference model. The point cloud data may be gathered until the data is determined to be sufficiently accurate for determining the relative positions of two or more teeth.

The relative position of the teeth may be computed by aligning the stitched point clouds generated by the progress scanning process to the segmented teeth surfaces and attachments in the reference model. During the alignment process accuracy of the gathered data for use in determining the degrees of freedom of the relative tooth positions may be determined using an optimization algorithm such as the iterative closest point algorithm, such as described above with respect to low and lower density intraoral scanning data, however, fewer frames of point cloud data may be gathered before reaching the desired accuracy, accordingly, this process may also be used with high or higher density scanners to speed up the subsequent scanning of a patient's dentition, such as during progress scanning.

The Iterative Closest Point (ICP) algorithm may be used to align and register two sets of 3D or 2D points to find their optimal spatial transformation (translation and rotation) that minimizes the differences between corresponding points in the two sets, sometime referred to as stitch two sets of point clouds together.

A first part of the process may include starting with an initial estimate of the transformation between the two point sets, such as the stitched point cloud data from the progress scan set and the reference model. This initial estimate can be obtained through various means, such as manual alignment or rough estimation or randomly.

Next, for each point in the source stitched point cloud set of the progress scan data, the closest point in the target point set, such as the reference model, is found. This step establishes correspondences between points in the two sets. In some embodiments, the Euclidean distance may be used.

Next, weights may be assigned to the point correspondences based on their proximity. Points that are closer together may be given higher weights, while points that are farther apart have lower weights. This step may help in giving more importance to accurate correspondences.

Using the correspondences, either weighted or unweighted, an estimate of a transformation (usually a combination of translation and rotation) that aligns the source cloud with the target cloud is generated. One common method to do this is the Singular Value Decomposition technique. The estimated transformation to the source cloud may then be updated, transforming it closer to the alignment of the target cloud.

The alignment estimate may be checked to determine whether the alignment estimate has converged to a position and orientation within a threshold of alignment. This may be based on a convergence criteria, such as the change in transformation parameters falling below a certain threshold between iterations.

If the alignment threshold criteria are not met, then the process repeats. In some embodiments, the point cloud data is iteratively aligned with the reference model until the threshold is met. If the threshold is not met such as after a set number of iterations, additional point cloud data sets may be generated from the at-home intraoral scan or as part of the progress scanning process and stitched into the stitched point could data. In some embodiments the additional set or sets of point cloud data may be gathered, the data may be aligned, and the alignment may be checked against the threshold.

Although the iterative closest point method is described herein other alignment methods may be used. The other alignment methods may include an optimization procedure that attempts to minimize a loss function such as the minimal some of distances between the point cloud data of the progress tracking scan and the reference data from the treatment plan. In some embodiments, such as those with a significant number of degrees of freedom, a nonlinear optimization may be used.

One or more of many threshold criteria for determining whether the data is sufficiently accurate for determining the relative positions of two teeth may be used. For example, the optimization algorithm such as the iterative closest point algorithm may be run from multiple different starting points in the range of the solutions from each starting point may be determined. If for all or a sufficient number of starting positions, the solutions range is below a threshold of accuracy of the degrees of freedom then sufficiently accurate scan data has been captured. In some embodiments, the optimization may be run multiple times but noise may be added to the point cloud data. If each of the data sets with noise added converge to within a range of accuracy sufficient for determining the relative positions of two or more teeth then sufficiently accurate scan data has been captured.

In some embodiments, the optimization may be performed multiple times, but data points randomly removed from the point cloud data for each run of the optimization. If each run of the optimization is within a range or predetermined threshold of accuracy than the desired accuracy and have been achieved. In some embodiments, a combination of the optimizations described herein may be performed and, if they all converge to within a threshold of accuracy sufficient for determining the relative position of two or more teeth, then a sufficient number of the data sets have been captured.

In some embodiments, rather than optimizing for the degrees of freedom between two or more teeth, the process of aligning the point cloud to the reference model may be performed on a tooth by tooth basis for each individual tooth.

In some embodiments, alternatively or in addition to the optimizations discussed above, a machine learning method may be used to determine whether the received scan data from the progress scanning processes sufficient to determine the relative positions of the patient's teeth. The machine learning model may be trained using input past successful and/or unsuccessful optimization data. Machine learning model may be trained using features or aspects of the successful and/or unsuccessful optimization data including the number of data points for each tooth for each frame of gathered point cloud data, an estimate of the noise within the three-dimensional data in the data points, the number of scans collected as part of the scanning process for a particular tooth or set of teeth, the surface region of the teeth within the scan, in the shape of the tooth or teeth being scanned. The neural network or other machine learning model may be trained to determine whether sufficiently accurate data has been gathered based on these features and aspects. The model may make the accuracy determination without reference to the reference model, but simply based on the features and aspects of the data collected. A higher threshold for accuracy determination using the machine learning or neural network models may be used as compared to the optimization models discussed herein.

In some embodiments, non-structured light data may be used for the progress tracking scanning. For example, if more than one camera is used to capture data of the patient's dentition then stereo three-dimensional modeling may be used to generate the positions of the teeth in the progress scan, such as stereoscopic imaging. Other methods may include photogrammetry using one or more cameras, SLAM imagery, or other methods, such as shape from shading, or other deep neural network methods.

As described herein, a process may be used for determining the sufficiency of the images for accurately determining the relative positions of the patient's teeth in the three-dimensional modeling described above.

For example, the images may be processed to generate or otherwise determine the surface normal for each tooth within an image or set of images and the corresponding three-dimensional model generated therefrom. The estimated surface normal from the generated model may be compared to the surface normals in the reference model. For example in alignment between the surface normals in the progress tracking data may be checked against the surface normals in the reference model and an optimization, such as described above, may be used to determine the accuracy of the image data based on the surface normals.

The relative position of the teeth may be computed by aligning surface models generated by the progress scanning process to the segmented teeth surfaces and attachments in reference model. During the alignment process accuracy of the gathered data for use in determining the degrees of freedom of the relative tooth positions may be determined using an optimization algorithm.

A first part of the process may include starting with an initial estimate of the transformation between the two surface normal sets, such as the progress scan set and the reference model. This initial estimate can be obtained through various means, such as manual alignment or rough estimation or randomly.

Next, for each surface normal in the source surface normal set of the progress scan data, the closest match of a surface normal in the target point set, such as the reference model, is found. This step establishes correspondences between service normals in the two sets. In some embodiments, the Euclidean distance may be used.

Next, weights may be assigned to the service normals based on their location. For example service normals in hard to estimate regions or locations on the teeth in the reference model may be given lower weight and service normals in easier to estimate regions or locations on the teeth.

Using the correspondences, either weighted or unweighted, an estimate of a transformation (usually a combination of translation and rotation) that aligns the source service normals with the target surface normals is generated.

The alignment estimate may be checked to determine whether the alignment estimate has converged to a position and orientation within a threshold of alignment. This may be based on a convergence criteria, such as the change in transformation parameters falling below a certain threshold between iterations.

If the alignment threshold criteria are not met, then the process repeats. In some embodiments, the surface normal data is iteratively aligned with the reference model until the threshold is met. If the threshold is not met such as after a set number of iterations, additional surface normal data sets may be generated from the at-home intraoral scan or as part of the progress scanning process. In some embodiments the additional set or sets of surface normal data may be gathered, the data may be aligned, and the alignment may be checked against the threshold.

Although the one method is described herein other methods may be used. The other alignment methods may include an optimization procedure that attempts to minimize a loss function such as the minimal some of distances between the surface normal data of the progress tracking scan and the reference data from the treatment plan. In some embodiments, such as those with a significant number of degrees of freedom, a nonlinear optimization may be used.

One or more of many threshold criteria for determining whether the data is sufficiently accurate for determining the relative positions of two teeth may be used. For example, the optimization algorithm may be run from multiple different starting points in the range of the solutions from each starting point may be determined. If for all or a sufficient number of starting positions, the solutions range is below a threshold of accuracy of the degrees of freedom then sufficiently accurate scan data has been captured. In some embodiments, the optimization may be run multiple times but noise may be added to the surface normal data. If each of the data sets with noise added converge to within a range of accuracy sufficient for determining the relative positions of two or more teeth then sufficiently accurate scan data has been captured.

In some embodiments, the optimization may be performed multiple times, but data points randomly removed from the surface normal data for each run of the optimization. If each run of the optimization is within a range or predetermined threshold of accuracy than the desired accuracy and have been achieved. In some embodiments, a combination of the optimizations described herein may be performed and, if they all converge to within a threshold of accuracy sufficient for determining the relative position of two or more teeth, then a sufficient number of the data sets have been captured.

In some embodiments different subsets of images may be used in the optimization algorithm in order to determine the best efficiency of the accuracy of the images for use in determining the relative positions of the teeth. If each of the subsets converges to the same result or within a threshold difference, then the images may be determined to be sufficiently accurate.

In some embodiments, rather than optimizing for the degrees of freedom between two or more teeth, the process of aligning the point cloud to the reference model may be performed on a tooth by tooth basis for each individual tooth.

The accuracy determination may be performed locally such as on the patient's device or on a remote computer or server. Once sufficient we accurate data has been received, feedback may be provided to the user.

At block 350 feedback may be provided to the user as to the scanning process. With reference to FIG. 5, the feedback may include highlighted areas 508 of the patient's teeth. The highlighted areas may indicate the portions of the patient's dentition that have been scanned sufficiently accurately for determining the relative positions of the patient's teeth. The highlighted portion may include coloring the teeth a color different than the non-scanned images. In some embodiments the feedback may also include the portion of the dentition currently being scanned. In some embodiments, attachments 504 may be located on the patient's dentition. Attachments identified in the scan data, such as attachment 504a, may be highlighted as part of the feedback. In some embodiments attachments identified in the scan data may be given a different color than attachments not yet identified in the scan data, such as attachments 504b. In some embodiments, attachments identified within the field of view of the scanner but not yet sufficiently identified may be highlighted, such as attachment 504c. As the progress tracking scan process progresses identified attachments may change in color from, for example, red to indicate not scanned, yellow to indicate currently being scanned, and a third color to indicate that the attachment has been identified in the scan data with sufficiently accurate scan data having been captured.

In some embodiments, during treatment, attachments may have fallen off of the patient's teeth. In such embodiments, if an attachment I was on a tooth wearing sufficient data has been captured to determine its position and orientation relative to the other teeth, but the attachment is not present in the scan data, then the attachment may be highlighted or colored with fourth color.

The process may proceed from block 350 back to block 330, for example, where the process repeats until the scan is complete. In some embodiments, the process may proceed from block 350 back to block 340, for example, where the process repeats until the scan is complete.

At block 360 the progress tracking scan is completed. For example, the progress scan may be complete when sufficiently accurate data for determining the relative positions of the patient's teeth for the upper and lower arch has been captured. In some embodiments, the progress scan may only capture a subset of the relative positions of the patient's teeth such as those expected to move in the teeth immediately surrounding the teeth that are expected to move based on the treatment plan. In such embodiments, the progress scan may be complete when data sufficient to determine the relative positions of the patient's teeth that are expected to move and the immediately adjacent teeth have been captured.

At block 370 the status of the dentition is determined. The process of determining the status the dentition may occur locally on the patient's device for remotely such as on a remote computer or server. Process of determining the status of the patient's dentition may include determining whether the relative positions of the patient's teeth in the progress scan match for our within a threshold deviation from the expected positions according to the treatment plan. In some embodiments, other diagnostic information may be determined and provided from the scan data such as gum recession, gingivitis, missing teeth, or other dental issues.

If the progress scanning is performed as part of a treatment tracking process such as at step 140, upon completion of the process, the process may proceed to for example block 150 of method 100 for further action with respect to the patient's dental treatment.

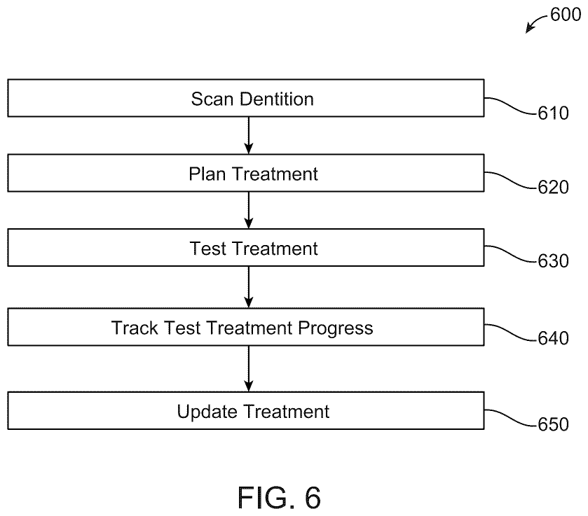

FIG. 6 shows a method 600 for dental treatment using remote progress scanning of a patient's teeth. The method 600 may include scanning a dentition of a patient at block 610, generating a treatment plan at block 620, testing the patient's response to treatment at block 630, tracking test treatment progress at block 640, and updating the treatment plan at block 650.

At block 610, in some embodiments, the patient's dentition is scanned with the three-dimensional intraoral scanner in order to generate a three-dimensional model of the patient's dentition. The three-dimensional model of the patient's dentition may be segmented to include individually segmented teeth representing each of the patient's teeth of their upper and lower arches.

For example, FIG. 2A depicts a three-dimensional model 200 of an arch of a patient's dentition. The three-dimensional model may include the shape, position, and orientation of the patient's teeth 202, and other dental and oral tissues within the oral cavity. For example the three-dimensional model may include the patient's gingiva, also referred to as gums, tongue, lips, and other anatomy. The initial scan of the patient's teeth 202 includes the position and orientation of the patient's teeth at the time of the scan.

At block 620 a treatment plan may be generated. An assessment may be made as to desired final positions of the patient's teeth based on the initial position of the patient's teeth obtained from the intraoral scan. A series of intermediate tooth positions of the patient's teeth may be generated to incrementally move the teeth through a series of stages from the initial positions towards the final positions.

In some embodiments, initial aligners may be generated to test the patient's response to treatment. For example, for teeth that are moved as part of the treatment plan may be moved, using test aligners, to determine the teeth's response to treatment, such as the rate they move in response to particular tooth movement forces.

FIG. 2B depicts a three-dimensional model 204 of an arch of the patient's dentition generated as part of the treatment planning process. The three-dimensional model may include the shape, position, and orientation of the patient's teeth 202 during a stage of treatment, and other dental and oral tissues within the oral cavity. In some embodiments individual attachments or other anchoring elements 104 may be used on teeth 202 with corresponding receptacles or apertures 1006 in the appliance so that the appliance can apply a selected force on the tooth. In some embodiments, a reference model as discussed herein may include the position and orientation of the patient's teeth and one or more attachments located thereon.

At block 630 a test treatment may begin. The test appliances may be fabricated to move the patient's teeth. A patient then wears each of the test appliances for a period of time, for example 10 days to two weeks, during which time the dental appliance applies forces to the patient's teeth to move the teeth from a first position at the beginning of the treatment stage towards a second position. Each test appliance is worn in succession in order to move the patient's teeth.

At block 640 progress of the test treatment and in particular is carried out to determine how the patient's teeth moved in response to the test aligners. Progress tracking may include comparing the actual movement and position of a patient's teeth during orthodontic treatment as compared to the expected movement and position of the patient's teeth during orthodontic treatment. The comparison may include determining the positional deviation or error between the actual position of the patient's teeth and the expected position of the patient's teeth based on a three-dimensional model of the patient's teeth for the particular stage of treatment.

The progress scanning may occur at a remote location such as a location remote from a dental office and may be performed using a lower quality scanner as compared to the scanner used to generate the initial treatment plan and 3D model. The lower quality scanner which may be referenced as a home intraoral scanner, may have a lower resolution and lower accuracy than the scanner used to generate the initial treatment planning a 3D model, which may be referred to as the reference scan or reference model. For example, the lower resolution may include using a lower density of data points when generating scan, such as between 0.5 and 4 megapixels, and preferably between 0.25 and 2 megapixels, for cameras and between six and 15 lines for structured light system. In some embodiments, the structured light system may capture less than 150 depth points within the field of view, preferably between 50 and 100 depth points within the field of view. As another example, the lower accuracy of the scanner may include using the scanner having a lower accuracy in the determination of the depth location of a data point.

When using a lower accuracy 3D scanner the data generated by the home intraoral scanner may be analyzed to detect when enough data from the home intraoral 3D scanner has been generated in order to determine the position of the particular tooth. The various processes and systems for performing the progress tracking scan are shown and described herein with reference to FIGS. 3-5.

At block 650 the treatment plan may be updated based on movement of the patient's teeth during the test portion of the treatment. For example, if the patient's teeth moved as fast as expected, then the treatment plan may proceed unmodified or, if no treatment plan had been generated, then the teeth may be moved and aligners worn for a typical amount of time. If the teeth moved slower than expected, then the treatment plan be modified to include more stages with smaller movements per stage, or the same number of stages, but in each stage the appliance is worn for a longer period of time. If the teeth moved faster than expected, then the treatment plan be modified to include fewer stages with larger movements per stage or the same number of stages, but in each stage the appliance is worn for a shorter period of time.

FIG. 7 depicts an embodiments, of a scanning system 700. As discussed herein, the scanning system 700 may include a low resolution scanner 710 that is coupled in electronic communication with a patient device 720. The low resolution scanner 710 that is coupled in electronic communication with a patient device 720 via wireless communication, such as WiFi or Bluetooth or other forms of wireless communication discussed herein or via wired communication, such as a USB, thunderbolt, or other forms of wired communication discussed herein.

The patient device may communicate with a remote computing system 730 and/or a dental office system 740, as discussed herein. Similarly, the remote computing system and dental office system may communicate with each other as discussed herein.

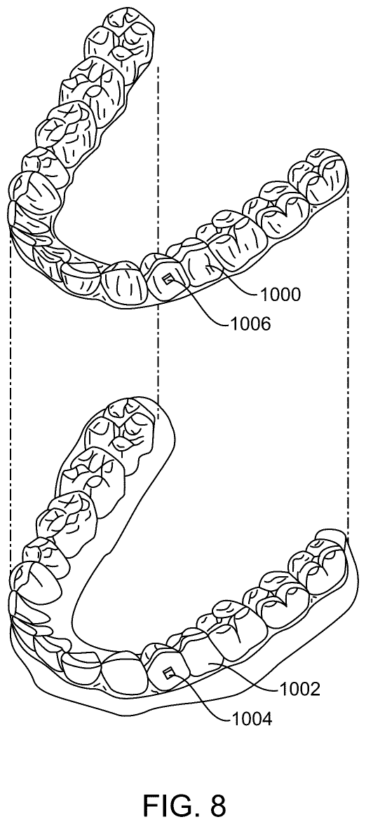

FIG. 8 illustrates an exemplary tooth repositioning appliance 1000, such as an aligner that can be worn by a patient in order to achieve an incremental repositioning of individual teeth 1002 in the jaw. The appliance can include a shell (e.g., a continuous polymeric shell or a segmented shell) having teeth-receiving cavities that receive and resiliently reposition the teeth. An appliance or portion(s) thereof may be indirectly fabricated using a physical model of teeth. For example, an appliance (e.g., polymeric appliance) can be formed using a physical model of teeth and a sheet of suitable layers of polymeric material. The physical model (e.g., physical mold) of teeth can be formed through a variety of techniques, including 3D printing. The appliance can be formed by thermoforming the appliance over the physical model. In some embodiments, a physical appliance is directly fabricated, e.g., using additive manufacturing techniques, from a digital model of an appliance. In some embodiments, the physical appliance may be created through a variety of direct formation techniques, such as 3D printing. An appliance can fit over all teeth present in an upper or lower jaw, or less than all of the teeth. The appliance can be designed specifically to accommodate the teeth of the patient (e.g., the topography of the tooth-receiving cavities matches the topography of the patient's teeth), and may be fabricated based on positive or negative models of the patient's teeth generated by impression, scanning, and the like. Alternatively, the appliance can be a generic appliance configured to receive the teeth, but not necessarily shaped to match the topography of the patient's teeth. In some cases, only certain teeth received by an appliance will be repositioned by the appliance while other teeth can provide a base or anchor region for holding the appliance in place as it applies force against the tooth or teeth targeted for repositioning. In some cases, some or most, and even all, of the teeth will be repositioned at some point during treatment. Teeth that are moved can also serve as a base or anchor for holding the appliance as it is worn by the patient. In some embodiments, no wires or other means will be provided for holding an appliance in place over the teeth. In some cases, however, it may be desirable or necessary to provide individual attachments or other anchoring elements 1004 on teeth 1002 with corresponding receptacles or apertures 1006 in the appliance 1000 so that the appliance can apply a selected force on the tooth. Exemplary appliances, including those utilized in the Invisalign® System, are described in numerous patents and patent applications assigned to Align Technology, Inc. including, for example, in U.S. Pat. Nos. 6,450,807, and 5,975,893, as well as on the company's website, which is accessible on the World Wide Web (see, e.g., the URL “invisalign.com”). Examples of tooth-mounted attachments suitable for use with orthodontic appliances are also described in patents and patent applications assigned to Align Technology, Inc., including, for example, U.S. Pat. Nos. 6,309,215 and 6,830,450.

FIG. 8 illustrates a tooth repositioning system 1100 including a plurality of appliances 1103A, 1103B, 1103C. Any of the appliances described herein can be designed and/or provided as part of a set of a plurality of appliances used in a tooth repositioning system. Each appliance may be configured so a tooth-receiving cavity has a geometry corresponding to an intermediate or final tooth arrangement intended for the appliance. The patient's teeth can be progressively repositioned from an initial tooth arrangement to a target tooth arrangement by placing a series of incremental position adjustment appliances over the patient's teeth. For example, the tooth repositioning system 1100 can include a first appliance 1103A corresponding to an initial tooth arrangement, one or more intermediate appliances 1103B corresponding to one or more intermediate arrangements, and a final appliance 1103C corresponding to a target arrangement. A target tooth arrangement can be a planned final tooth arrangement selected for the patient's teeth at the end of all planned orthodontic treatment. Alternatively, a target arrangement can be one of some intermediate arrangements for the patient's teeth during the course of orthodontic treatment, which may include various different treatment scenarios, including, but not limited to, instances where surgery is recommended, where interproximal reduction (IPR) is appropriate, where a progress check is scheduled, where anchor placement is best, where palatal expansion is desirable, where restorative dentistry is involved (e.g., inlays, onlays, crowns, bridges, implants, veneers, and the like), etc. As such, it is understood that a target tooth arrangement can be any planned resulting arrangement for the patient's teeth that follows one or more incremental repositioning stages. Likewise, an initial tooth arrangement can be any initial arrangement for the patient's teeth that is followed by one or more incremental repositioning stages.

Optionally, in cases involving more complex movements or treatment plans, it may be beneficial to utilize auxiliary components (e.g., features, accessories, structures, devices, components, and the like) in conjunction with an orthodontic appliance. Examples of such accessories include but are not limited to elastics, wires, springs, bars, arch expanders, palatal expanders, twin blocks, occlusal blocks, bite ramps, mandibular advancement splints, bite plates, pontics, hooks, brackets, headgear tubes, springs, bumper tubes, palatal bars, frameworks, pin-and-tube apparatuses, buccal shields, buccinator bows, wire shields, lingual flanges and pads, lip pads or bumpers, protrusions, divots, and the like. In some embodiments, the appliances, systems and methods described herein include improved orthodontic appliances with integrally formed features that are shaped to couple to such auxiliary components, or that replace such auxiliary components.

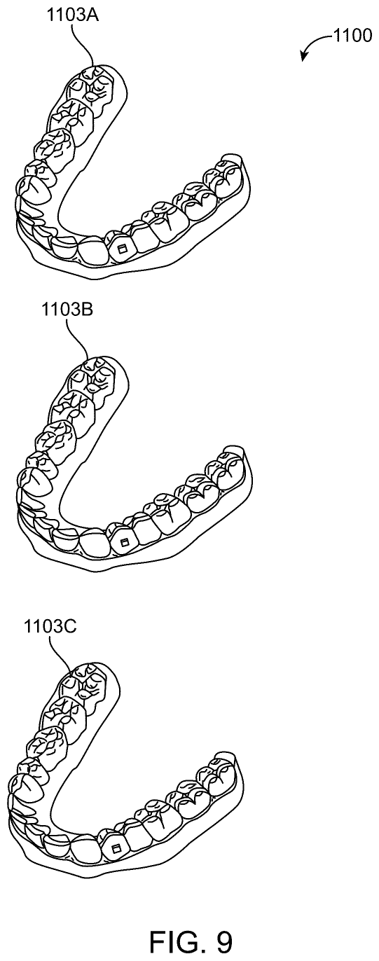

FIG. 9 illustrates a method 1200 of orthodontic treatment using a plurality of appliances, in accordance with many embodiments. The method 1200 can be practiced using any of the appliances or appliance sets described herein. In step 1210, a first orthodontic appliance is applied to a patient's teeth in order to reposition the teeth from a first tooth arrangement to a second tooth arrangement. In step 1220, a second orthodontic appliance is applied to the patient's teeth in order to reposition the teeth from the second tooth arrangement to a third tooth arrangement. The method 1200 can be repeated as necessary using any suitable number and combination of sequential appliances in order to incrementally reposition the patient's teeth from an initial arrangement to a target arrangement. The appliances can be generated all at the same stage or in sets or batches (e.g., at the beginning of a stage of the treatment), or one at a time, and the patient can wear each appliance until the pressure of each appliance on the teeth can no longer be felt or until the maximum amount of expressed tooth movement for that given stage has been achieved. A plurality of different appliances (e.g., a set) can be designed and even fabricated prior to the patient wearing any appliance of the plurality. After wearing an appliance for an appropriate period of time, the patient can replace the current appliance with the next appliance in the series until no more appliances remain. The appliances are generally not affixed to the teeth and the patient may place and replace the appliances at any time during the procedure (e.g., patient-removable appliances). The final appliance or several appliances in the series may have a geometry or geometries selected to overcorrect the tooth arrangement. For instance, one or more appliances may have a geometry that would (if fully achieved) move individual teeth beyond the tooth arrangement that has been selected as the “final.” Such over-correction may be desirable in order to offset potential relapse after the repositioning method has been terminated (e.g., permit movement of individual teeth back toward their pre-corrected positions). Over-correction may also be beneficial to speed the rate of correction (e.g., an appliance with a geometry that is positioned beyond a desired intermediate or final position may shift the individual teeth toward the position at a greater rate). In such cases, the use of an appliance can be terminated before the teeth reach the positions defined by the appliance. Furthermore, over-correction may be deliberately applied in order to compensate for any inaccuracies or limitations of the appliance.

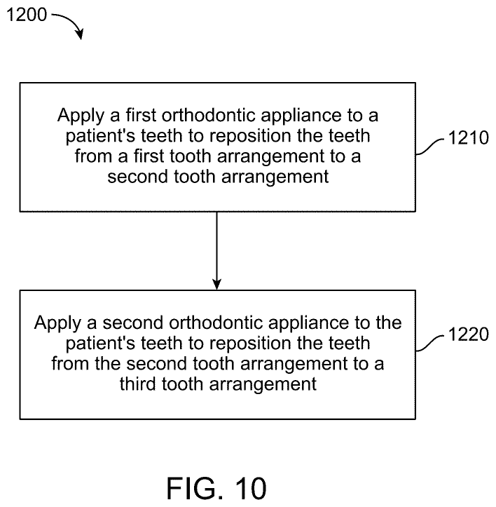

FIG. 10 illustrates a method 1300 for digitally planning an orthodontic treatment and/or design or fabrication of an appliance, in accordance with many embodiments. The method 1300 can be applied to any of the treatment procedures described herein and can be performed by any suitable data processing system. Any embodiment of the appliances described herein can be designed or fabricated using the method 1300.

In step 1310, a digital representation of a patient's teeth is received. The digital representation can include surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.).

In step 1320, one or more treatment stages are generated based on the digital representation of the teeth. The treatment stages can be incremental repositioning stages of an orthodontic treatment procedure designed to move one or more of the patient's teeth from an initial tooth arrangement to a target arrangement. For example, the treatment stages can be generated by determining the initial tooth arrangement indicated by the digital representation, determining a target tooth arrangement, and determining movement paths of one or more teeth in the initial arrangement necessary to achieve the target tooth arrangement. The movement path can be optimized based on minimizing the total distance moved, preventing collisions between teeth, avoiding tooth movements that are more difficult to achieve, or any other suitable criteria.

In step 1330, at least one orthodontic appliance is fabricated based on the generated treatment stages. For example, a set of appliances can be fabricated to be sequentially worn by the patient to incrementally reposition the teeth from the initial arrangement to the target arrangement. Some of the appliances can be shaped to accommodate a tooth arrangement specified by one of the treatment stages. Alternatively or in combination, some of the appliances can be shaped to accommodate a tooth arrangement that is different from the target arrangement for the corresponding treatment stage. For example, as previously described herein, an appliance may have a geometry corresponding to an overcorrected tooth arrangement. Such an appliance may be used to ensure that a suitable amount of force is expressed on the teeth as they approach or attain their desired target positions for the treatment stage. As another example, an appliance can be designed in order to apply a specified force system on the teeth and may not have a geometry corresponding to any current or planned arrangement of the patient's teeth.

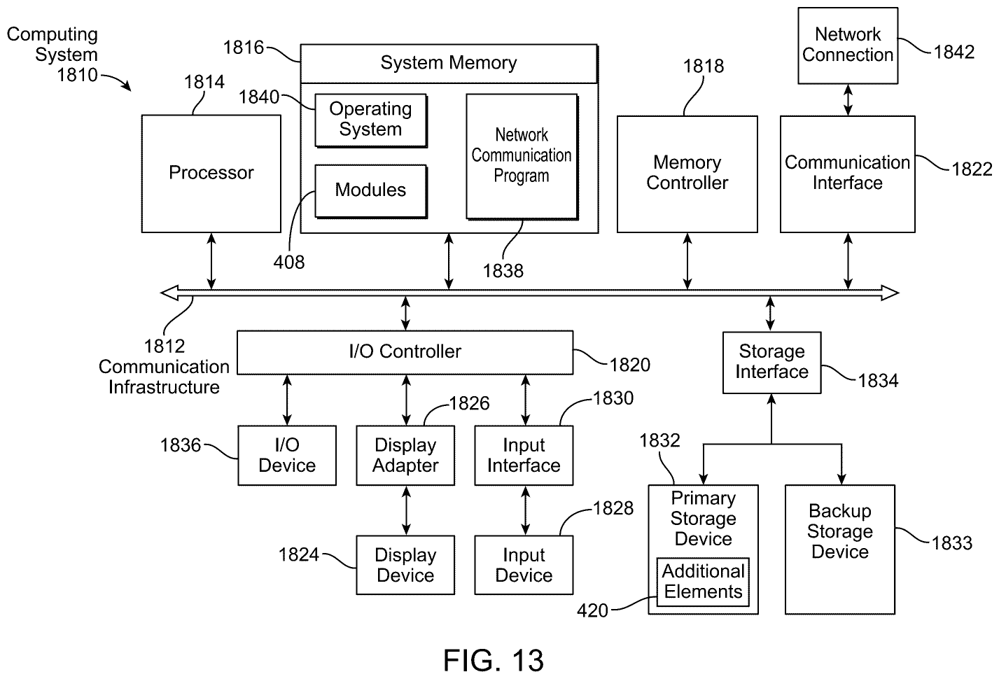

In some instances, staging of various arrangements or treatment stages may not be necessary for design and/or fabrication of an appliance. As illustrated by the dashed line in FIG. 13, design and/or fabrication of an orthodontic appliance, and perhaps a particular orthodontic treatment, may include use of a representation of the patient's teeth (e.g., receive a digital representation of the patient's teeth 1310), followed by design and/or fabrication of an orthodontic appliance based on a representation of the patient's teeth in the arrangement represented by the received representation.

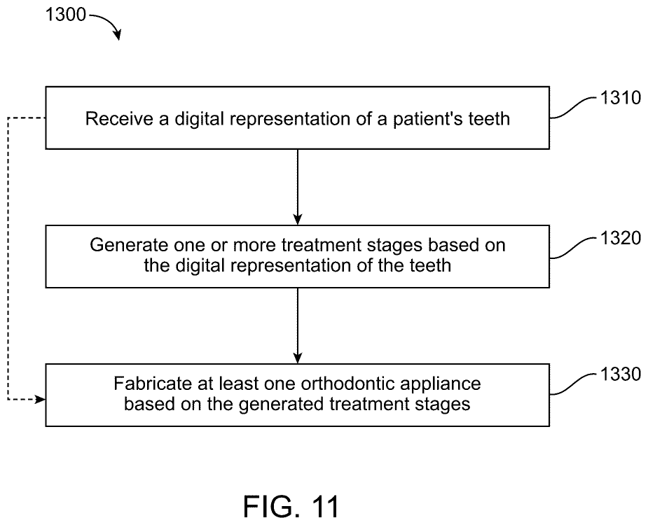

FIG. 11 is a simplified block diagram of a data processing system 1400 that may be used in executing methods and processes described herein. The data processing system 1400 typically includes at least one processor 1402 that communicates with one or more peripheral devices via bus subsystem 1404. These peripheral devices typically include a storage subsystem 1406 (memory subsystem 1408 and file storage subsystem 1414), a set of user interface input and output devices 1418, and an interface to outside networks 1416. This interface is shown schematically as “Network Interface” block 1416, and is coupled to corresponding interface devices in other data processing systems via communication network interface 1424. Data processing system 1400 can include, for example, one or more computers, such as a personal computer, workstation, mainframe, laptop, and the like.

The user interface input devices 1418 are not limited to any particular device, and can typically include, for example, a keyboard, pointing device, mouse, scanner, interactive displays, touchpad, joysticks, etc. Similarly, various user interface output devices can be employed in a system of the invention, and can include, for example, one or more of a printer, display (e.g., visual, non-visual) system/subsystem, controller, projection device, audio output, and the like.

Storage subsystem 1406 maintains the basic required programming, including computer readable media having instructions (e.g., operating instructions, etc.), and data constructs. The program modules discussed herein are typically stored in storage subsystem 1406. Storage subsystem 1406 typically includes memory subsystem 1408 and file storage subsystem 1414. Memory subsystem 1408 typically includes a number of memories (e.g., RAM 1410, ROM 1412, etc.) including computer readable memory for storage of fixed instructions, instructions and data during program execution, basic input/output system, etc. File storage subsystem 1414 provides persistent (non-volatile) storage for program and data files, and can include one or more removable or fixed drives or media, hard disk, floppy disk, CD-ROM, DVD, optical drives, and the like. One or more of the storage systems, drives, etc. may be located at a remote location, such coupled via a server on a network or via the internet/World Wide Web. In this context, the term “bus subsystem” is used generically so as to include any mechanism for letting the various components and subsystems communicate with each other as intended and can include a variety of suitable components/systems that would be known or recognized as suitable for use therein. It will be recognized that various components of the system can be, but need not necessarily be at the same physical location, but could be connected via various local-area or wide-area network media, transmission systems, etc.

Scanner 1420 includes any means for obtaining a digital representation (e.g., images, surface topography data, etc.) of a patient's teeth (e.g., by scanning physical models of the teeth such as casts 1421, by scanning impressions taken of the teeth, or by directly scanning the intraoral cavity), which can be obtained either from the patient or from treating professional, such as an orthodontist, and includes means of providing the digital representation to data processing system 1400 for further processing. Scanner 1420 may be located at a location remote with respect to other components of the system and can communicate image data and/or information to data processing system 1400, for example, via a network interface 1424. Fabrication system 1422 fabricates appliances 1423 based on a treatment plan, including data set information received from data processing system 1400. Fabrication machine 1422 can, for example, be located at a remote location and receive data set information from data processing system 1400 via network interface 1424.

### Computing System

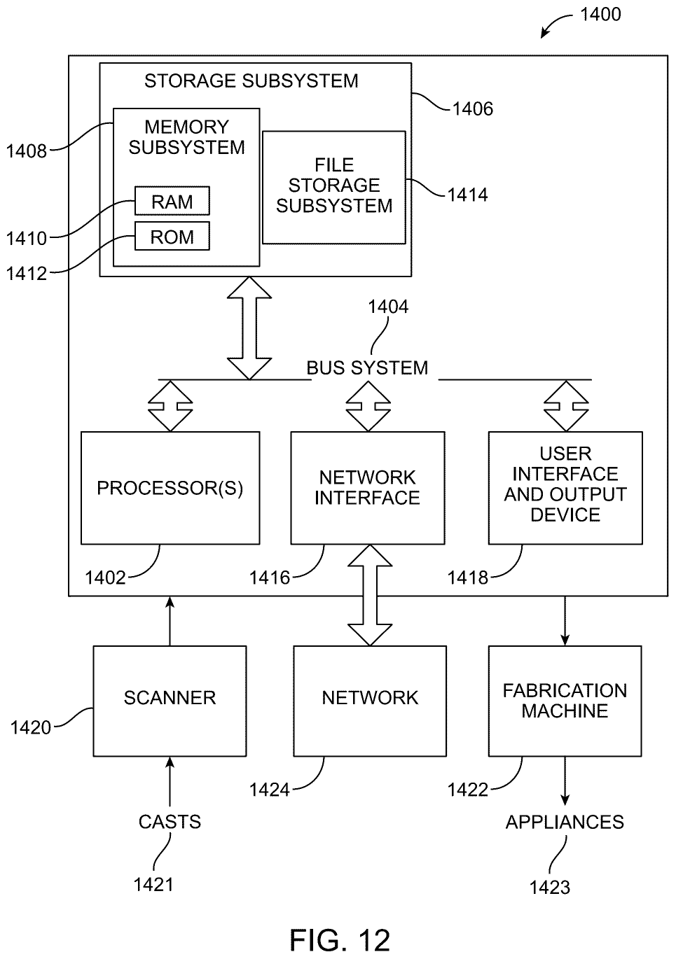

FIG. 12 is a block diagram of an example computing system 1810 capable of implementing one or more of the embodiments described and/or illustrated herein. For example, all or a portion of computing system 1810 may perform and/or be a means for performing, either alone or in combination with other elements, one or more of the steps described herein (such as one or more of the steps illustrated in FIGS. 1, 3, 6, and 10). All or a portion of computing system 1810 may also perform and/or be a means for performing any other steps, methods, or processes described and/or illustrated herein.

Computing system 1810 broadly represents any single or multi-processor computing device or system capable of executing computer-readable instructions. Examples of computing system 1810 include, without limitation, workstations, laptops, client-side terminals, servers, distributed computing systems, handheld devices, or any other computing system or device. In its most basic configuration, computing system 1810 may include at least one processor 1814 and a system memory 1816.

Processor 1814 generally represents any type or form of physical processing unit (e.g., a hardware-implemented central processing unit) capable of processing data or interpreting and executing instructions. In certain embodiments, processor 1814 may receive instructions from a software application or module. These instructions may cause processor 1814 to perform the functions of one or more of the example embodiments described and/or illustrated herein.

System memory 1816 generally represents any type or form of volatile or non-volatile storage device or medium capable of storing data and/or other computer-readable instructions. Examples of system memory 1816 include, without limitation, Random Access Memory (RAM), Read Only Memory (ROM), flash memory, or any other suitable memory device. Although not required, in certain embodiments computing system 1810 may include both a volatile memory unit (such as, for example, system memory 1816) and a non-volatile storage device (such as, for example, primary storage device 1832, as described in detail below). In one example, one or more of the software components or instructions described herein may be loaded into system memory 1816.

In some examples, system memory 1816 may store and/or load an operating system 1840 for execution by processor 1814. In one example, operating system 1840 may include and/or represent software that manages computer hardware and software resources and/or provides common services to computer programs and/or applications on computing system 1810. Examples of operating system 1840 include, without limitation, LINUX, JUNOS, MICROSOFT WINDOWS, WINDOWS MOBILE, MAC OS, APPLE'S IOS, UNIX, GOOGLE CHROME OS, GOOGLE'S ANDROID, SOLARIS, variations of one or more of the same, and/or any other suitable operating system.

In certain embodiments, example computing system 1810 may also include one or more components or elements in addition to processor 1814 and system memory 1816. For example, as illustrated in FIG. 12, computing system 1810 may include a memory controller 1818, an Input/Output (I/O) controller 1820, and a communication interface 1822, each of which may be interconnected via a communication infrastructure 1812. Communication infrastructure 1812 generally represents any type or form of infrastructure capable of facilitating communication between one or more components of a computing device. Examples of communication infrastructure 1812 include, without limitation, a communication bus (such as an Industry Standard Architecture (ISA), Peripheral Component Interconnect (PCI), PCI Express (PCIe), or similar bus) and a network.

Memory controller 1818 generally represents any type or form of device capable of handling memory or data or controlling communication between one or more components of computing system 1810. For example, in certain embodiments memory controller 1818 may control communication between processor 1814, system memory 1816, and I/O controller 1820 via communication infrastructure 1812.

I/O controller 1820 generally represents any type or form of module capable of coordinating and/or controlling the input and output functions of a computing device. For example, in certain embodiments I/O controller 1820 may control or facilitate transfer of data between one or more elements of computing system 1810, such as processor 1814, system memory 1816, communication interface 1822, display adapter 1826, input interface 1830, and storage interface 1834.

As illustrated in FIG. 12, computing system 1810 may also include at least one display device 1824 coupled to I/O controller 1820 via a display adapter 1826. Display device 1824 generally represents any type or form of device capable of visually displaying information forwarded by display adapter 1826. Similarly, display adapter 1826 generally represents any type or form of device configured to forward graphics, text, and other data from communication infrastructure 1812 (or from a frame buffer, as known in the art) for display on display device 1824.

As illustrated in FIG. 12, example computing system 1810 may also include at least one input device 1828 coupled to I/O controller 1820 via an input interface 1830. Input device 1828 generally represents any type or form of input device capable of providing input, either computer or human generated, to example computing system 1810. Examples of input device 1828 include, without limitation, a keyboard, a pointing device, a speech recognition device, variations or combinations of one or more of the same, and/or any other input device.

Additionally or alternatively, example computing system 1810 may include additional I/O devices. For example, example computing system 1810 may include I/O device 1836. In this example, I/O device 1836 may include and/or represent a user interface that facilitates human interaction with computing system 1810. Examples of I/O device 1836 include, without limitation, a computer mouse, a keyboard, a monitor, a printer, a modem, a camera, a scanner, a microphone, a touchscreen device, variations or combinations of one or more of the same, and/or any other I/O device.

Communication interface 1822 broadly represents any type or form of communication device or adapter capable of facilitating communication between example computing system 1810 and one or more additional devices. For example, in certain embodiments communication interface 1822 may facilitate communication between computing system 1810 and a private or public network including additional computing systems. Examples of communication interface 1822 include, without limitation, a wired network interface (such as a network interface card), a wireless network interface (such as a wireless network interface card), a modem, and any other suitable interface. In at least one embodiment, communication interface 1822 may provide a direct connection to a remote server via a direct link to a network, such as the Internet. Communication interface 1822 may also indirectly provide such a connection through, for example, a local area network (such as an Ethernet network), a personal area network, a telephone or cable network, a cellular telephone connection, a satellite data connection, or any other suitable connection.

In certain embodiments, communication interface 1822 may also represent a host adapter configured to facilitate communication between computing system 1810 and one or more additional network or storage devices via an external bus or communications channel. Examples of host adapters include, without limitation, Small Computer System Interface (SCSI) host adapters, Universal Serial Bus (USB) host adapters, Institute of Electrical and Electronics Engineers (IEEE) 1394 host adapters, Advanced Technology Attachment (ATA), Parallel ATA (PATA), Serial ATA (SATA), and External SATA (eSATA) host adapters, Fibre Channel interface adapters, Ethernet adapters, or the like. Communication interface 1822 may also allow computing system 1810 to engage in distributed or remote computing. For example, communication interface 1822 may receive instructions from a remote device or send instructions to a remote device for execution.

In some examples, system memory 1816 may store and/or load a network communication program 1838 for execution by processor 1814. In one example, network communication program 1838 may include and/or represent software that enables computing system 1810 to establish a network connection 1842 with another computing system (not illustrated in FIG. 12) and/or communicate with the other computing system by way of communication interface 1822. In this example, network communication program 1838 may direct the flow of outgoing traffic that is sent to the other computing system via network connection 1842. Additionally or alternatively, network communication program 1838 may direct the processing of incoming traffic that is received from the other computing system via network connection 1842 in connection with processor 1814.

Although not illustrated in this way in FIG. 12, network communication program 1838 may alternatively be stored and/or loaded in communication interface 1822. For example, network communication program 1838 may include and/or represent at least a portion of software and/or firmware that is executed by a processor and/or Application Specific Integrated Circuit (ASIC) incorporated in communication interface 1822.

As illustrated in FIG. 12, example computing system 1810 may also include a primary storage device 1832 and a backup storage device 1833 coupled to communication infrastructure 1812 via a storage interface 1834. Storage devices 1832 and 1833 generally represent any type or form of storage device or medium capable of storing data and/or other computer-readable instructions. For example, storage devices 1832 and 1833 may be a magnetic disk drive (e.g., a so-called hard drive), a solid state drive, a floppy disk drive, a magnetic tape drive, an optical disk drive, a flash drive, or the like. Storage interface 1834 generally represents any type or form of interface or device for transferring data between storage devices 1832 and 1833 and other components of computing system 1810. In one example, one or more of the software components or instructions described herein may be stored and/or loaded in primary storage device 1832.

In certain embodiments, storage devices 1832 and 1833 may be configured to read from and/or write to a removable storage unit configured to store computer software, data, or other computer-readable information. Examples of suitable removable storage units include, without limitation, a floppy disk, a magnetic tape, an optical disk, a flash memory device, or the like. Storage devices 1832 and 1833 may also include other similar structures or devices for allowing computer software, data, or other computer-readable instructions to be loaded into computing system 1810. For example, storage devices 1832 and 1833 may be configured to read and write software, data, or other computer-readable information. Storage devices 1832 and 1833 may also be a part of computing system 1810 or may be a separate device accessed through other interface systems.

Many other devices or subsystems may be connected to computing system 1810. Conversely, all of the components and devices illustrated in FIG. 12 need not be present to practice the embodiments described and/or illustrated herein. The devices and subsystems referenced above may also be interconnected in different ways from that shown in FIG. 12. Computing system 1810 may also employ any number of software, firmware, and/or hardware configurations. For example, one or more of the example embodiments disclosed herein may be encoded as a computer program (also referred to as computer software, software applications, computer-readable instructions, or computer control logic) on a computer-readable medium. The term “computer-readable medium,” as used herein, generally refers to any form of device, carrier, or medium capable of storing or carrying computer-readable instructions. Examples of computer-readable media include, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

The computer-readable medium containing the computer program may be loaded into computing system 1810. All or a portion of the computer program stored on the computer-readable medium may then be stored in system memory 1816 and/or various portions of storage devices 1832 and 1833. When executed by processor 1814, a computer program loaded into computing system 1810 may cause processor 1814 to perform and/or be a means for performing the functions of one or more of the example embodiments described and/or illustrated herein. Additionally or alternatively, one or more of the example embodiments described and/or illustrated herein may be implemented in firmware and/or hardware. For example, computing system 1810 may be configured as an Application Specific Integrated Circuit (ASIC) adapted to implement one or more of the example embodiments disclosed herein.

FIG. 13 is a block diagram of an example network architecture 1900 in which client systems 1910, 1920, and 1930 and servers 1940 and 1945 may be coupled to a network 1950. As detailed above, all or a portion of network architecture 1900 may perform and/or be a means for performing, either alone or in combination with other elements, one or more of the steps disclosed herein (such as one or more of the steps illustrated in FIGS. 1, 3, 6, and 10). All or a portion of network architecture 1900 may also be used to perform and/or be a means for performing other steps and features set forth in the instant disclosure.

Client systems 1910, 1920, and 1930 generally represent any type or form of computing device or system, such as example computing system 1810 in FIG. 12. Similarly, servers 1940 and 1945 generally represent computing devices or systems, such as application servers or database servers, configured to provide various database services and/or run certain software applications. Network 1950 generally represents any telecommunication or computer network including, for example, an intranet, a WAN, a LAN, a PAN, or the Internet. In one example, client systems 1910, 1920, and/or 1930 and/or servers 1940 and/or 1945 may include all or a portion of the systems described in FIG. 4.

As illustrated in FIG. 13, one or more storage devices 1960(1)-(N) may be directly attached to server 1940. Similarly, one or more storage devices 1970(1)-(N) may be directly attached to server 1945. Storage devices 1960(1)-(N) and storage devices 1970(1)-(N) generally represent any type or form of storage device or medium capable of storing data and/or other computer-readable instructions. In certain embodiments, storage devices 1960(1)-(N) and storage devices 1970(1)-(N) may represent Network-Attached Storage (NAS) devices configured to communicate with servers 1940 and 1945 using various protocols, such as Network File System (NFS), Server Message Block (SMB), or Common Internet File System (CIFS).

Servers 1940 and 1945 may also be connected to a Storage Area Network (SAN) fabric 1980. SAN fabric 1980 generally represents any type or form of computer network or architecture capable of facilitating communication between a plurality of storage devices. SAN fabric 1980 may facilitate communication between servers 1940 and 1945 and a plurality of storage devices 1990(1)-(N) and/or an intelligent storage array 1995. SAN fabric 1980 may also facilitate, via network 1950 and servers 1940 and 1945, communication between client systems 1910, 1920, and 1930 and storage devices 1990(1)-(N) and/or intelligent storage array 1995 in such a manner that devices 1990(1)-(N) and array 1995 appear as locally attached devices to client systems 1910, 1920, and 1930. As with storage devices 1960(1)-(N) and storage devices 1970(1)-(N), storage devices 1990(1)-(N) and intelligent storage array 1995 generally represent any type or form of storage device or medium capable of storing data and/or other computer-readable instructions.

In certain embodiments, and with reference to example computing system 1810 of FIG. 12, a communication interface, such as communication interface 1822 in FIG. 12, may be used to provide connectivity between each client system 1910, 1920, and 1930 and network 1950. Client systems 1910, 1920, and 1930 may be able to access information on server 1940 or 1945 using, for example, a web browser or other client software. Such software may allow client systems 1910, 1920, and 1930 to access data hosted by server 1940, server 1945, storage devices 1960(1)-(N), storage devices 1970(1)-(N), storage devices 1990(1)-(N), or intelligent storage array 1995. Although FIG. 13 depicts the use of a network (such as the Internet) for exchanging data, the embodiments described and/or illustrated herein are not limited to the Internet or any particular network-based environment.

In at least one embodiment, all or a portion of one or more of the example embodiments disclosed herein may be encoded as a computer program and loaded onto and executed by server 1940, server 1945, storage devices 1960(1)-(N), storage devices 1970(1)-(N), storage devices 1990(1)-(N), intelligent storage array 1995, or any combination thereof. All or a portion of one or more of the example embodiments disclosed herein may also be encoded as a computer program, stored in server 1940, run by server 1945, and distributed to client systems 1910, 1920, and 1930 over network 1950.

As detailed above, computing system 1810 and/or one or more components of network architecture 1900 may perform and/or be a means for performing, either alone or in combination with other elements, one or more steps of an example method for virtual care.

While the foregoing disclosure sets forth various embodiments using specific block diagrams, flowcharts, and examples, each block diagram component, flowchart step, operation, and/or component described and/or illustrated herein may be implemented, individually and/or collectively, using a wide range of hardware, software, or firmware (or any combination thereof) configurations. In addition, any disclosure of components contained within other components should be considered example in nature since many other architectures can be implemented to achieve the same functionality.

In some examples, all or a portion of the systems described in FIGS. 11 and 12 may represent portions of a cloud-computing or network-based environment. Cloud-computing environments may provide various services and applications via the Internet. These cloud-based services (e.g., software as a service, platform as a service, infrastructure as a service, etc.) may be accessible through a web browser or other remote interface. Various functions described herein may be provided through a remote desktop environment or any other cloud-based computing environment.

In various embodiments, all or a portion of systems described in FIGS. 11 and 12 may facilitate multi-tenancy within a cloud-based computing environment. In other words, the software modules described herein may configure a computing system (e.g., a server) to facilitate multi-tenancy for one or more of the functions described herein. For example, one or more of the software modules described herein may program a server to enable two or more clients (e.g., customers) to share an application that is running on the server. A server programmed in this manner may share an application, operating system, processing system, and/or storage system among multiple customers (i.e., tenants). One or more of the modules described herein may also partition data and/or configuration information of a multi-tenant application for each customer such that one customer cannot access data and/or configuration information of another customer.

According to various embodiments, all or a portion of the systems described in FIGS. 11 and 12 may be implemented within a virtual environment. For example, the modules and/or data described herein may reside and/or execute within a virtual machine. As used herein, the term “virtual machine” generally refers to any operating system environment that is abstracted from computing hardware by a virtual machine manager (e.g., a hypervisor). Additionally or alternatively, the modules and/or data described herein may reside and/or execute within a virtualization layer. As used herein, the term “virtualization layer” generally refers to any data layer and/or application layer that overlays and/or is abstracted from an operating system environment. A virtualization layer may be managed by a software virtualization solution (e.g., a file system filter) that presents the virtualization layer as though it were part of an underlying base operating system. For example, a software virtualization solution may redirect calls that are initially directed to locations within a base file system and/or registry to locations within a virtualization layer.

In some examples, all or a portion of the systems described in FIGS. 11 and 12 may represent portions of a mobile computing environment. Mobile computing environments may be implemented by a wide range of mobile computing devices, including mobile phones, tablet computers, e-book readers, personal digital assistants, wearable computing devices (e.g., computing devices with a head-mounted display, smartwatches, etc.), and the like. In some examples, mobile computing environments may have one or more distinct features, including, for example, reliance on battery power, presenting only one foreground application at any given time, remote management features, touchscreen features, location and movement data (e.g., provided by Global Positioning Systems, gyroscopes, accelerometers, etc.), restricted platforms that restrict modifications to system-level configurations and/or that limit the ability of third-party software to inspect the behavior of other applications, controls to restrict the installation of applications (e.g., to only originate from approved application stores), etc. Various functions described herein may be provided for a mobile computing environment and/or may interact with a mobile computing environment.

In addition, all or a portion of the systems described in FIGS. 11 and 12 may represent portions of, interact with, consume data produced by, and/or produce data consumed by one or more systems for information management. As used herein, the term “information management” may refer to the protection, organization, and/or storage of data. Examples of systems for information management may include, without limitation, storage systems, backup systems, archival systems, replication systems, high availability systems, data search systems, virtualization systems, and the like.

In some embodiments, all or a portion of the systems described in FIGS. 11 and 12 may represent portions of, produce data protected by, and/or communicate with one or more systems for information security. As used herein, the term “information security” may refer to the control of access to protected data. Examples of systems for information security may include, without limitation, systems providing managed security services, data loss prevention systems, identity authentication systems, access control systems, encryption systems, policy compliance systems, intrusion detection and prevention systems, electronic discovery systems, and the like.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations, or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and shall have the same meaning as the word “comprising.

The processor as disclosed herein can be configured with instructions to perform any one or more steps of any method as disclosed herein.

It will be understood that although the terms “first,” “second,” “third,” etc. may be used herein to describe various layers, elements, components, regions, or sections without referring to any particular order or sequence of events. These terms are merely used to distinguish one layer, element, component, region or section from another layer, element, component, region, or section. A first layer, element, component, region, or section as described herein could be referred to as a second layer, element, component, region, or section without departing from the teachings of the present disclosure.

As used herein, the term “or” is used inclusively to refer items in the alternative and in combination.

As used herein, characters such as numerals refer to like elements.

The present disclosure includes the following numbered clauses.

Clause 1. A system for scanning a dentition using an at-home intraoral scanner, the system comprising: an intraoral scanner; and one or more processors and one or more memory comprising instructions that when executed by the one or more processors cause the system to: receive point cloud data; measure a range of possible models based on the received point cloud data by: manipulating an aspect of the point cloud data multiple times; for each manipulation of the point cloud data, calculating a possible model based on the altered point cloud data to generate a plurality of possible models; and computing a variance between each of the calculated possible models of the generated plurality of possible models; and output an indication to the user based on the measured range of possible models.

Clause 2. The system of clause 1, further comprising: a patient mobile device configured to be coupled in electronic communication with the intraoral scanner and comprising a first of the one or more processors; and a 3D processing system that is remote to the patient mobile device and comprising a second of the one or more processors, wherein the instructions that when executed by the one or more processors cause the first of the one or more processors to receive the point cloud data from the intraoral scanner and send the point cloud data to the 3D processing system, and wherein the instructions that when executed by the one or more processors cause the second of the one or more processors to measure a range of possible models based on the received point cloud data and send data for an indication based on the measured rage to the patient mobile device, and wherein the instructions that when executed by the one or more processors cause the first of the one or more processors to output an indication to the user based the data for the indication.

Clause 3. The system of clause 1, further comprising: a patient mobile device configured to be coupled in electronic communication with the intraoral scanner and comprising a first of the one or more processors, wherein the instructions that when executed by the one or more processors cause the first of the one or more processors to: receive the point cloud data; measure the range of possible models based on the received point cloud data by: manipulating the aspect of the point cloud data multiple times; for each manipulation of the point cloud data, calculating the possible model based on the altered point cloud data to generate the plurality of possible models; and computing the variance between each of the calculated possible models of the generated plurality of possible models; and output the indication to the user based on the measured range of possible models.

Clause 4. A system for scanning a dentition using an at-home intraoral scanner, the system comprising: a patient mobile device comprising a first one or more processors; an intraoral scanner configured to be coupled in electronic communication with the patient mobile device; a 3D processing system that is remote to the patient mobile device and comprising a second of the one or more processors, memory comprising instructions that when executed by the first and second one or more processors cause the system to: receive point cloud data by the mobile device from the intraoral scanner; measure a range of possible models based on the received point cloud data at the 3D processing system by: manipulating an aspect of the point cloud data multiple times; for each manipulation of the point cloud data, calculating a possible model based on the altered point cloud data to generate a plurality of possible models; and computing a variance between each of the calculated possible models of the generated plurality of possible models; and output an indication to the user based on the measured range of possible models by the patient mobile device.

Clause 5. The system of any one of clauses 1-4, wherein the range of possible models includes a plurality of tooth positions.

Clause 6. The system of any one of clauses 1-4, wherein manipulating the point cloud data comprises adding noise to the received point cloud data.

Clause 7. The system of any one of clauses 1-4, wherein manipulating the point cloud data comprises deleting points of the received point cloud data.

Clause 8. The system of any one of clauses 1-4, wherein manipulating the point cloud data comprises altering a starting position of the point cloud.

Clause 9. The system of any one of clauses 1-4, wherein each of the plurality of possible models comprises a relative positions of two neighboring teeth.

Clause 10. The system of any one of clauses 1-4, wherein the plurality of possible models is a transformation for each neighboring teeth with a variance on each parameter of the transformation.

Clause 11. The system of any one of clauses 1-4, wherein the instructions that when executed by the one or more processes further cause the system to: receive a type of treatment, based on a treatment plan; determine a predefined accuracy associated with the received type of treatment; compare the measured range of possible models to the predefined accuracy associated with the received type of treatment; and wherein the indication outputted is when the predefined accuracy is not reached.

Clause 12. The system of any one of clauses 1-4, wherein the instructions that when executed by the one or more processes further cause the system to: receive a type of treatment, based on a treatment plan; determine a predefined accuracy associated with the received type of treatment; compare the measured range of possible models to the predefined accuracy associated with the received type of treatment; and wherein the indication outputted is when the predefined accuracy is reached.

Clause 13. The system of any one of clauses 1-4, wherein the instructions that when executed by the one or more processes further cause the system to: receive a reference model based on a treatment plan, and wherein measuring a range of possible models based on the received point cloud data includes a comparison of the point cloud data with the reference model.

Clause 14. The method of clause 26, wherein the reference model was generated with a first intraoral scanner having a first resolution and accuracy and the point cloud data is generated with a second intraoral scanner having a second resolution and accuracy.

Clause 15. A method of using an intraoral scanner to scan a patient's dentition, the method comprising: receiving point cloud data; measuring a range of possible models based on the received point cloud data by: manipulating an aspect of the point cloud data multiple times; for each manipulation of the point cloud data, calculating a possible model based on the altered point cloud data to generate a plurality of possible models; and computing a variance between each of the calculated possible models of the generated plurality of possible models; and outputting an indication to the user based on the measured range of possible models.

Clause 16. A method of using an intraoral scanner to scan a patient's dentition, the method comprising: receiving point cloud data; measuring a range of possible models based on the received point cloud data until a target accuracy is reached; and outputting indications to the user based on the measured range of possible models during the measuring of the range of possible models.

Clause 17. The method of clause 16, wherein measuring the range of possible models based on the received point cloud data until the target accuracy is reached includes: manipulating an aspect of the point cloud data multiple times; for each manipulation of the point cloud data, calculating a possible model based on the altered point cloud data to generate a plurality of possible models; and computing a variance between each of the calculated possible models of the generated plurality of possible models; and

Clause 18. The method of any one of clause 15-17, wherein the range of possible models includes a plurality of tooth positions and shapes.

Clause 19. The method of any one of clause 15-17, wherein manipulating the point cloud data comprises adding noise to the received point cloud data.

Clause 20. The method of any one of clause 15-17, wherein manipulating the point cloud data comprises deleting points of the received point cloud data.

Clause 21. The method of any one of clause 15-17, wherein manipulating the point cloud data comprises altering a starting position of the point cloud.

Clause 22. The method of any one of clause 15-17, wherein each of the plurality of possible models comprises a relative positions of two neighboring teeth.

Clause 23. The method of any one of clause 15-17, wherein the plurality of possible models is a transformation for each neighboring teeth with a variance on each parameter of the transformation.

Clause 24. The method of any one of clause 15-17, further comprising: receiving a type of treatment, based on a treatment plan; determining a predefined accuracy associated with the received type of treatment; comparing the measured range of possible models to the predefined accuracy associated with the received type of treatment; and wherein the indication outputted is when the predefined accuracy is not reached.

Clause 25. The method of any one of clause 15-17, further comprising: receiving a type of treatment, based on a treatment plan; determining a predefined accuracy associated with the received type of treatment; comparing the measured range of possible models to the predefined accuracy associated with the received type of treatment; and wherein the indication outputted is when the predefined accuracy is reached.

Clause 26. The method of any one of clause 15-17, further comprising: receiving a reference model based on a treatment plan, and wherein measuring a range of possible models based on the received point cloud data includes a comparison of the point cloud data with the reference model.

Clause 27. The method of clause 26, wherein the reference model was generated with a first intraoral scanner having a first resolution and accuracy and the point cloud data is generated with a second intraoral scanner having a second resolution and accuracy, the first resolution and accuracy being greater than the second resolution and accuracy.

Clause 28. A method of using an intraoral scanner to scan a patient's dentition, the method comprising: receiving image data; measuring a range of possible models based on the received image data by: generating 3D data from the image data; manipulating an aspect of the 3D data multiple times; for each manipulation of the 3D data, calculating a possible model based on the altered 3D data to generate a plurality of possible models; and computing a variance between each of the calculated possible models of the generated plurality of possible models; and outputting an indication to the user based on the measured range of possible models.

Clause 29. The method of clause 28, wherein the 3D data includes surface normal data of the surfaces within the image.

Clause 30. The method of clause 28, wherein manipulating the 3D data comprises adding noise to the received 3D data.

Clause 31. The method of clause 28, wherein manipulating the 3D data comprises deleting points of the received 3D data.

Clause 32. The method of clause 28, wherein manipulating the point cloud data comprises altering a starting position of the 3D data.

Clause 33. The method of clause 28, wherein each of the plurality of possible models comprises a relative positions of two neighboring teeth.

Clause 34. The method of clause 28, wherein the plurality of possible models is a transformation for each neighboring teeth with a variance on each parameter of the transformation.

Clause 35. The method of clause 28, further comprising: receiving a type of treatment, based on a treatment plan; determining a predefined accuracy associated with the received type of treatment; comparing the measured range of possible models to the predefined accuracy associated with the received type of treatment; and wherein the indication outputted is when the predefined accuracy is not reached.

Clause 36. The method of clause 28, further comprising: receiving a type of treatment, based on a treatment plan; determining a predefined accuracy associated with the received type of treatment; comparing the measured range of possible models to the predefined accuracy associated with the received type of treatment; and wherein the indication outputted is when the predefined accuracy is reached.

Clause 37. The method of clause 28, further comprising: receiving a reference model based on a treatment plan, and wherein measuring a range of possible models based on the received point cloud data includes a comparison of the point cloud data with the reference model.

Clause 38. The method of clause 37, wherein the reference model was generated with a first intraoral scanner having a first resolution and accuracy and the point cloud data is generated with a second intraoral scanner having a second resolution and accuracy.

Embodiments of the present disclosure have been shown and described as set forth herein and are provided by way of example only. One of ordinary skill in the art will recognize numerous adaptations, changes, variations, and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

What is claimed is:

1. A system for scanning a dentition using an at-home intraoral scanner, the system comprising:
an intraoral scanner; and
one or more processors and one or more memory comprising instructions that when executed by the one or more processors cause the system to:
receive point cloud data of a patient's detention from the intraoral scanner during an intraoral scanning process;
generate, during the intraoral scanning process, a plurality of possible models of the patient's dentition based on the received point cloud data by:
manipulating an aspect of the point cloud data multiple times;
for each manipulation of the point cloud data, generating a possible model of the patient's dentition based on the manipulated point cloud data to generate the plurality of possible models of the patient's dentition; and
determining a position variance between positions of respective dental features of the patient's detention between each of the possible models of the of the patient's dentition; and
output a depiction of the scanned location on the patient's detention based the measured range of possible models.

2. The system of claim 1, further comprising:
a patient mobile device configured to be coupled in electronic communication with the intraoral scanner and comprising a first of the one or more processors; and
a 3D processing system that is remote to the patient mobile device and comprising a second of the one or more processors,
wherein the instructions that when executed by the one or more processors cause the first of the one or more processors to receive the point cloud data from the intraoral scanner and send the point cloud data to the 3D processing system, and
wherein the instructions that when executed by the one or more processors cause the second of the one or more processors to measure a range of possible models based on the received point cloud data and send data for an indication based on the measured rage to the patient mobile device, and
wherein the instructions that when executed by the one or more processors cause the first of the one or more processors to output an indication to the user based the data for the indication.

3. The system of claim 1, further comprising:
a patient mobile device configured to be coupled in electronic communication with the intraoral scanner and comprising a first of the one or more processors,
wherein the instructions that when executed by the one or more processors cause the first of the one or more processors to:
receive the point cloud data;
generate a plurality of possible models based on the received point cloud data by:
manipulating the aspect of the point cloud data multiple times;
for each manipulation of the point cloud data, calculating the possible model based on the manipulated point cloud data to generate the plurality of possible models; and
computing the variance between each of the calculated possible models of the generated plurality of possible models; and
output the depiction of the scanned location on the patient's detention based on the measured range of possible models.

4. A system for scanning a dentition using an at-home intraoral scanner, the system comprising:
a patient mobile device comprising a first one or more processors;
an intraoral scanner configured to be coupled in electronic communication with the patient mobile device;
a 3D processing system that is remote to the patient mobile device and comprising a second of the one or more processors,
memory comprising instructions that when executed by the first and second one or more processors cause the system to:
receive point cloud data of a patient's detention by the mobile device from the intraoral scanner;
generating, during the intraoral scanning process, a plurality of possible models based on the received point cloud data at the 3D processing system by:
manipulating an aspect of the point cloud data multiple times;
for each manipulation of the point cloud data, possible model of the patient's dentition based on the manipulated point cloud data to generate the plurality of possible models of the patient's dentition;
computing a variance between positions of respective dental features of the patient's detention between each of the possible models of the of the patient's dentition; and
output a depiction of the scanned location on the patient's detention based the measured range of possible models.

5. The system of claim 4, wherein the plurality of possible models includes a plurality of tooth positions.

6. The system of claim 4, wherein manipulating the point cloud data comprises adding noise to the received point cloud data.

7. The system of claim 4, wherein manipulating the point cloud data comprises deleting points of the received point cloud data.

8. The system of claim 4, wherein manipulating the point cloud data comprises altering a starting position of the point cloud.

9. The system of claim 4, wherein each of the plurality of possible models comprises a relative positions of two neighboring teeth.

10. The system of claim 4, wherein the plurality of possible models is a transformation for each neighboring teeth with a variance on each parameter of the transformation.

11. The system of claim 4, wherein the instructions that when executed by the one or more processes further cause the system to:
receive a type of treatment, based on a treatment plan;
determine a predefined accuracy associated with the received type of treatment;
compare the measured range of possible models to the predefined accuracy associated with the received type of treatment; and
wherein the indication is output when the predefined accuracy is not reached.

12. The system of claim 4, wherein the instructions that when executed by the one or more processes further cause the system to:
receive a type of treatment, based on a treatment plan;
determine a predefined accuracy associated with the received type of treatment;
compare the measured range of possible models to the predefined accuracy associated with the received type of treatment; and
wherein the indication is output when the predefined accuracy is reached.

13. The system of claim 4, wherein the instructions that when executed by the one or more processes further cause the system to:
receive a reference model based on a treatment plan, and wherein measuring a range of possible models based on the received point cloud data includes a comparison of the point cloud data with the reference model.

14. The method of claim 13, wherein the reference model was generated with a first intraoral scanner having a first resolution and accuracy and the point cloud data is generated with a second intraoral scanner having a second resolution and accuracy.

15. A method of using an intraoral scanner to scan a patient's dentition, the method comprising:
receiving point cloud data;
measuring a range of possible models based on the received point cloud data by:
manipulating an aspect of the point cloud data multiple times;
for each manipulation of the point cloud data, calculating a possible model based on the manipulated point cloud data to generate a plurality of possible models; and
computing a variance between each of the calculated possible models of the generated plurality of possible models; and
outputting an indication to the user based on the measured range of possible models.

16. The method of claim 15, wherein the range of possible models includes a plurality of tooth positions and shapes.

17. The method of claim 15, wherein manipulating the point cloud data comprises adding noise to the received point cloud data.

18. The method of claim 15, wherein manipulating the point cloud data comprises deleting points of the received point cloud data.

19. The method of any one of claim 15, further comprising:
receiving a reference model based on a treatment plan, and wherein measuring a range of possible models based on the received point cloud data includes a comparison of the point cloud data with the reference model.

20. The method of claim 19, wherein the reference model was generated with a first intraoral scanner having a first resolution and accuracy and the point cloud data is generated with a second intraoral scanner having a second resolution and accuracy, the first resolution and accuracy being greater than the second resolution and accuracy.
